(19) United States
(12) Patent Application Publication
(10) Pub. No.: US 2004/0253598 A1
(43) Pub. Date: Dec. 16, 2004

(54) Vesicle-associated proteins

(76) Inventors: Mariah R. Baughn, Los Angeles, CA (US); Ernestine A. Lee, Kensington, CA (US); Vicki S. Elliott, San Jose, CA (US); Brendan M. Duggan, Sunnyvale, CA (US); Joana X. Li, Millbrae, CA (US); Jennifer A. Griffin, Fremont, CA (US); April J.A. Hafalia, Daly City, CA (US); Angelo M. Delegeane, Milpitas, CA (US); Soo Yeun Lee, Mountain View, CA (US); Jayalaxmi Ramkumar, Fremont, CA (US); Amy E. Kable, Silver Spring, MD (US); Joseph P. Marquis, San Jose, CA (US); Rajagopal Gururajan, San Jose, CA (US); William W. (Webb) Sprague, Sacramento, CA (US); Junming Yang, San Jose, CA (US); Kimberly J. Gietzen, San Jose, CA (US); Yeganeh Zebarjadian, San Francisco, CA (US); Thomas W. Richardson, Redwood City, CA (US); Alan A. Jackson, Los Gatos, CA (US); Xin Jiang, Saratoga, CA (US); Shanya D. Becha, San Francisco, CA (US)

Correspondence Address: INCYTE CORPORATION, EXPERIMENTAL STATION, ROUTE 141 & HENRY CLAY ROAD, BLDG. E336, WILMINGTON, DE, 19880, US

(21) Appl. No.: 10/491,471

(22) PCT Filed: Oct. 24, 2002
(86) PCT No.: PCT/US02/34452

(30) Foreign Application Priority Data
Oct. 26, 2001 (US) 60347927
Nov. 13, 2001 (US) 60332908
Nov. 20, 2001 (US) 60331865
Dec. 20, 2001 (US) 60342604
Feb. 6, 2002 (US) 60354827

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

Various embodiments of the invention provide human vesicle-associated proteins (VAP) and polynucleotides which identify and encode VAP. Embodiments of the invention also provide expression vectors, host cells, antibodies, agonists, and antagonists. Other embodiments provide methods for diagnosing, treating, or preventing disorders associated with aberrant expression of VAP.

### TECHNICAL FIELD

The invention relates to novel nucleic acids, vesicle-associated proteins encoded by these nucleic acids, and to the use of these nucleic acids and proteins in the diagnosis, treatment, and prevention of vesicle trafficking disorders, autoimmune/inflammatory disorders, and cancer. The invention also relates to the assessment of the effects of exogenous compounds on the expression of nucleic acids and vesicle-associated proteins.

### BACKGROUND OF THE INVENTION

Eukaryotic cells are bound by a lipid bilayer membrane and subdivided into functionally distinct, membrane-bound compartments. The membranes maintain the essential differences between the cytosol, the extracellular environment, and the lumenal space of each intracellular organelle. As lipid membranes are highly impermeable to most polar molecules, transport of essential nutrients, metabolic waste products, cell signaling molecules, macromolecules, and proteins across lipid membranes and between organelles must be mediated by a variety of transport-associated molecules.

Integral membrane proteins, secreted proteins, and proteins destined for the lumen of organelles are synthesized within the endoplasmic reticulum (ER), delivered to the Golgi complex for post-translational processing and sorting, and then transported to specific intracellular and extracellular destinations. Material is internalized from the extracellular environment by endocytosis, a process essential for transmission of neuronal, metabolic, and proliferative signals; uptake of many essential nutrients; and defense against invading organisms. This intracellular and extracellular movement of protein molecules is termed vesicle trafficking. Trafficking is accomplished by the packaging of protein molecules into specialized vesicles which bud from the donor organelle membrane and fuse to the target membrane (Rothman, J. E and F. T. Wieland (1996) Science 272:227-234).

The transport of proteins across the ER membrane involves a process that is simnilar in bacteria, yeast, and mammals (Gorlich, D. et al. (1992) Cell 71:489-503). In mammalian systems, transport is initiated by the action of a cytoplasmic signal recognition particle (SRP) which recognizes a signal sequence on a growing, nascent polypeptide and binds the polypeptide and its ribosome complex to the ER membrane through an SRP receptor located on the ER membrane. The signal peptide is cleaved and the ribosome complex, together with the attached polypeptide, becomes membrane bound. The polypeptide is subsequently translocated across the ER membrane and into a vesicle (Blobel, G. and B. Dobberstein (1975) J. Cell Biol. 67:852-862).

Proteins implicated in the translocation of polypeptides across the ER membrane in yeast include SEC61p, SEC62p, and SEC63p. Mutations in the genes encoding these proteins lead to defects in the translocation process. SEC61 may be of particular importance since certain mutations in the gene for this protein inhibit the translocation of many proteins (Gorlich et al., supra).

Marrmalian homologs of yeast SEC61 (mSEC61) have been identified in dog and rat (Gorlich et al., supra). Mammalian SEC61 is also structurally similar to SECYp, the bacterial cytoplasmic membrane translocation protein. mSEC61 is found in tight association with membrane-bound ribosomes. This association is induced by membrane-targeting of nascent polypeptide chains and is weakened by dissociation of the ribosomes into their constituent subunits. mSEC61 is postulated to be a component of a putative protein-conducting channel, located in the ER membrane, to which nascent polypeptides are transferred following the completion of translation by ribosomes (Gorlich et al., supra).

Several steps in the transit of material along the secretory and endocytic pathways require the formation of transport vesicles. Specifically, vesicles form at the transitional endoplasmic reticulum (tER), the rim of Golgi cisternae, the face of the Trans-Golgi Network (TGN), the plasma membrane (PM), and tubular extensions of the endosomes. Vesicle formation occurs when a region of membrane buds off from the donor organelle. The membrane-bound vesicle contains proteins to be transported and is surrounded by a proteinaceous coat, the components of which are recruited from the cytosol. Vesicle formation begins with the budding of a vesicle out of a donor organelle. The initial budding and coating processes are controlled by a cytosolic ras-like GTP-binding protein, ADP-ribosylating factor (Arf), and adapter proteins (APs). Different isoforms of both Arf and AP are involved at different sites of budding. For example, Arfs 1, 3, and 5 are required for Golgi budding, Arf4 for endosomal budding, and Arf6 for plasma membrane budding. Two different classes of coat protein have also been identified. Clathrin coats form on vesicles derived from the TGN and PM, whereas coatomer (COP) coats form on vesicles derived from the ER and Golgi (Mellman, I. (1996) Annu. Rev. Cell Dev. Biol. 12:575625).

In clathrin-based vesicle formation, APs bring vesicle cargo and coat proteins together at the surface of the budding membrane. APs are heterotetrameric complexes composed of two large chains (α, γ, δ, or ε, and β), a medium chain (μ), and a small chain (σ). Clathrin binds to APs via the carboxy-terminal appendage domain of the β-adaptin subunit (Le Bourgne, R. and B. Hoflack (1998) Curr. Opin. Cell. Biol. 10:499-503). AP-1 functions in protein sorting from the TGN and endosomes to compartments of the endosomal/lysosomal system. AP-2 functions in clathrin-mediated endocytosis at the plasma membrane, while AP-3 is associated with endosomes and/or the TGN and recruits integral membrane proteins for transport to lysosomes and lysosome-related organelles. The recently isolated AP-4 complex localizes to the TGN or a neighboring compartment and may play a role in sorting events thought to take place in post-Golgi compartments (Dell'Angelica, E. C. et al. (1999) J. Biol. Chem. 274:7278-7285). Cytosolic GTP-bound Arf is also incorporated into the vesicle as it forms. Another GTP-binding protein, dynamin, forms a ring complex around the neck of the forming vesicle and provides the mechanochemical force required to release the vesicle from the donor membrane. The coated vesicle complex is then transported through the cytosol. During the transport process, Arf-bound GTP is hydrolyzed to GDP and the coat dissociates from the transport vesicle (West, M. A. et al. (1997) J. Cell Biol. 138:1239-1254).

Coatomer (COP) coats form on vesicles derived from the ER and Golgi. COP coats can further be distinguished as COPI, involved in retrograde traffic through the Golgi to the ER, and COPII, involved in anterograde traffic from the ER to the Golgi. The COP coat consists of two major components, a GTP-binding protein (Arf or Sar) and coat protomer (coatomer). Coatomer is an equimolar complex of seven proteins, termed alpha-, beta-, beta′-, gamma-, delta-, epsilon- and zeta-COP. The coatomer complex binds to dilysine motifs contained on the cytoplasmic tails of integral membrane proteins. These include the dilysine-containing retrieval motif of membrane proteins of the ER and dibasic/diphenylamine motifs of members of the p24 family. The p24 family of type I membrane proteins represent the major membrane proteins of COPI vesicles (Harter, C. and F. T. Wieland (1998) Proc. Natl. Acad. Sci. USA 95:11649-11654).

Vesicles can undergo homotypic or heterotypic fusion. Molecules required for appropriate targeting and fusion of vesicles include proteins in the vesicle membrane, the target membrane, and proteins recruited from the cytosol. During budding of the vesicle from the donor compartment, an integral membrane protein, VAMP (vesicle-associated membrane protein) is incorporated into the vesicle. Soon after the vesicle uncoats, a cytosolic prenylated GTP-binding protein, Rab, is inserted into the vesicle membrane. In the vesicle membrane, GTP-bound Rab interacts with VAMP.

The amino acid sequences of Rab proteins reveal conserved GTP-binding domains characteristic of Ras superfamnily members. Rab proteins also have a highly variable amino terminus containing membrane-specific signal information and a prenylated carboxy terminus which determines the target membrane to which the Rab proteins anchor. More than 30 Rab proteins have been identified in a variety of species, and each has a characteristic intracellular location and distinct transport function. In particular, Rab1 and Rab2 are important in ER-to-Golgi transport; Rab3 transports secretory vesicles to the extracellular membrane; Rab5 is localized to endosomes and regulates the fusion of early endosomes into late endosomes; Rab6 is specific to the Golgi apparatus and regulates intra-Golgi transport events; Rab7 and Rab9 stimulate the fusion of late endosomes and Golgi vesicles with lysosomes, respectively; and Rab10 mediates vesicle fusion from the medial Golgi to the trans Golgi. Mutant forms of Rab proteins are able to block protein transport along a given pathway or alter the sizes of entire organelles. Therefore, Rabs play key regulatory roles in membrane trafficking (Schimmoller, I. S. and S. R. Pfeffer (1998) J. Biol. Chem. 243:22161-22164).

The function of Rab proteins in vesicular transport requires the cooperation of many other proteins. Specifically, the membrane-targeting process is assisted by a series of escort proteins (Khosravi-Par, R. et al. (1991) Proc. Natl. Acad. Sci. USA 88:6264-6268). In the medial Golgi, it has been shown that GTP-bound Rab proteins initiate the binding of VAMP-like proteins of the transport vesicle to syntaxin-like proteins on the acceptor membrane, which subsequently triggers a cascade of protein-binding and membrane-fusion events. After transport, GTPase-activating proteins (GAPs) in the target membrane are responsible for converting the GTP-bound Rab proteins to their GDP-bound state. And finally a cytosolic protein, guanine-nucleotide dissociation inhibitor (GDI), removes GDP-bound Rab from the vesicle membrane.

Docking of the transport vesicle with the target membrane involves the formation of a complex between the vesicle SNAP receptor (v-SNARE), target membrane (t-) SNAREs, and certain other membrane and cytosolic proteins. Many of these other proteins have been identified although their exact functions in the docking complex remain uncertain (Tellam, J. T. et al. (1995) J. Biol. Chem. 270:5857-5863; Hata, Y. and T. C. Sudhof (1995) J. Biol. Chem. 270:13022-13028). N-ethylmaleimide sensitive factor (NSF) and soluble NSF-attachment protein (α-SNAP and β-SNAP) are two such proteins that are conserved from yeast to man and function in most intracellular membrane fusion reactions. Many of these membrane and cytosolic proteins contain an AAA protein family signature domain. The AAA protein family signature consists of a large family of ATPases whose key feature is that they share a conserved region of approximately 200 amino acids that contains an ATP-binding site. This family is called AAA, for ‘A’ TPases ‘A’ ssociated with diverse cellular ‘A’ ctivities. The proteins that belong to this family either contain one or two AAA domains. Mammalian NSF contains two AAA domains, involved in intracellular transport between the endoplasmic reticulum and Golgi, as well as between different Golgi cisternae. Secl represents a family of yeast proteins that function at many different stages in the secretory pathway including membrane fusion. Recently, mammalian homologs of Sec I, called Munc-18 proteins, have been identified (Katagiri, H. et al. (1995) J. Biol. Chem. 270:49634966; Hata and Sudhof, supra). Sec22p is a yeast v-SNARE required for transport between the ER and the Golgi apparatus. Marnmalian sec22 homologs have been identified in humans, rats, mice, and hamsters (Tang, B. L. et al. (1998) Biochem. Biophys. Res. Commun. 243:885-91; and references within).

The SNARE complex involves three SNARE molecules, one in the vesicular membrane and two in the target membrane. Together they form a rod-shaped complex of four α-helical coiled-coils. The membrane anchoring domains of all three SNAREs project from one end of the rod. This complex is similar to the rod-like structures formed by fusion proteins characteristic of the enveloped viruses, such as myxovirus, influenza, filovirus (Ebola), and the HW and SIV retroviruses (Skehel, J. J. and D. C. Wiley (1998) Cell 95:871-874). It has been proposed that the SNARE complex is sufficient for membrane fusion, suggesting that the proteins which associate with the complex provide regulation over the fusion event (Weber, T. et al. (1998) Cell 92:759-772). For example, in neurons, which exhibit regulated exocytosis, docked vesicles do not fuse with the presynaptic membrane until depolarization, which leads to an influx of calcium (Bennett, M. K. and R. H. Scheller (1994) Annu. Rev. Biochem. 63:63-100). Synaptotagmin, an integral membrane protein in the synaptic vesicle, associates with the t-SNARE syntaxin in the docking complex. Synaptotagmin binds calcium in a complex with negatively charged phospholipids, which allows the cytosolic SNAP protein to displace synaptotagmin from syntaxin and fusion to occur. Thus, synaptotagmin is a negative regulator of fusion in the neuron (Littleton, J. T. et al. (1993) Cell 74:1125-1134). The most abundant membrane protein of synaptic vesicles appears to be the glycoprotein synaptophysin, a 38 kDa protein with four transmembrane domains. Although the function of synaptophysin is not known, its calcium-binding ability, tyrosine phosphorylation, and widespread distribution in neural tissues suggest a potential role in neurosecretion (Bennett and Scheller, supra). The synaptojanin family of proteins have been implicated in synaptic vesicle recycling and actin function. Synaptojanins are phosphoinositide phosphatases predominantly expressed in the nervous system. One form of synaptojanin, synaptojanin 2A, is targeted to mitochondria by the interaction with the PDZ-domain of a mitochondrial outer membrane protein (Nemoto, Y. and P. De Camilli (1999) EMBO J. 18:2991-3006).

The transport of proteins into and out of vesicles relies on interactions between cell membranes and a supporting membrane cytoskeleton consisting of spectrin and other proteins. A large family of related proteins called ankyrins participate in the transport process by binding to the membrane skeleton protein spectrin and to a protein in the cell membrane called band 3, a component of an anion channel in the cell membrane. Ankyrins therefore function as a critical link between the cytoskeleton and the cell membrane.

Originally found in association with erythroid cells, ankyrins are also found in other tissues as well (Birkenmeier, C. S. et al. (1993) J. Biol. Chem. 268:9533-9540). Ankyrins are large proteins (1800 amino acids) containing an N-terminal, 89 kDa domain that binds the cell membrane proteins band 3 and tubulin, a central 62 kDa domain that binds the cytoskeletal proteins spectrin and vimentin, and a C-terminal, 55 kDa regulatory domain that functions as a modifier of the binding activities of the other two domains. Individual genes for ankyrin are able to produce multiple ankyrin isoforms by various insertions and deletions. These isoforms are of nearly identical size but may have different functions. In addition, smaller transcripts are produced which are missing large regions of the coding sequences from the N-terminal (band 3 binding), and central (spectrin binding) domains. The existence of such a large family of ankyrin proteins and the observation that more than one type of ankyrin may be expressed in the same cell type suggests that ankyrins may have more specialized functions than simply binding the membrane skeleton to the plasma membrane (Birkenmeier et al., supra).

In humans, two isoforms of ankyrin are expressed, alternatively, in developing erythroids and mature erythroids, respectively (Lambert, S. et. al. (1990) Proc. Natl. Acad. Sci. USA 87:1730-1734). A deficiency in erythroid spectrin and ankyrin has been associated with the hemolytic anemia, hereditary spherocytosis (Coetzer, T. L. et al. (1988) New Engl. J. Med. 318:230-234).

Correct trafficking of proteins is of particular importance for the proper function of epithelial cells, which are polarized into distinct apical and basolateral domains containing different cell membrane components such as lipids and membrane-associated proteins. Certain proteins are flexible and may be sorted to the basolateral or apical side depending upon cell type or growth conditions. For example, the kidney anion exchanger (kAE1) can be retargeted from the apical to the basolateral domain if cells are plated at higher density. The protein kanadaptin was isolated as a protein which binds to the cytoplasmic domain of kAE1. It also colocalizes with kAE1 in vesicles, but not in the membrane, suggesting that kanadaptin's function is to guide kAE1-containing vesicles to the basolateral target membrane (Chen, J. et al. (1998) J. Biol. Chem. 273:1038-1043).

Vesicle trafficking is crucial in the process of neurotransmission. Synaptic vesicles carry neurotransmitter molecules from the cytoplasm of a neuron to the synapse. Rab3s are a family of GTP-binding proteins located on synaptic vesicles. The RIM family of proteins are thought to be effectors for Rab3s (Wang, Y. et al. (2000) J. Biol. Chem. 275:20033-20044). Rabphilin-3 is a synaptic vesicle protein. Granuphilins are proteins with homology to rabphilins, and may have a unique role in exocytosis (Wang, J. et al. (1999) J. Biol. Chem. 274:28542-28548).

As studied in nematodes, vesicle-associated proteins are also involved in sperm motility. Major sperm protein (MSP) contributes to sperm pseudopodial movement by forming a cytosolic filament network that translocates vesicles to the plasma membrane (Italiano, J. E. et al. (1996) Cell 84:105-114; Roberts, T. M. et al. (1998) J. Cell Biol. 140:367-75).

The etiology of numerous human diseases and disorders can be attributed to defects in the trafficking of proteins to organelles or the cell surface. Defects in the trafficking of membrane-bound receptors and ion channels are associated with cystic fibrosis (cystic fibrosis transmembrane conductance regulator; CFTR), glucose-galactose malabsorption syndrome (Na⁺/glucose cotransporter), hypercholesterolemia (low-density lipoprotein (LDL) receptor), and forms of diabetes mellitus (insulin receptor). Abnormal hormonal secretion is linked to disorders including diabetes insipidus (vasopressin), hyper- and hypoglycemia (insulin, glucagon), Grave's disease and goiter (thyroid hormone), and Cushing's and Addison's diseases (adrenocorticotropic hormone; ACT1H).

Cancer cells secrete excessive amounts of hormones or other biologically active peptides. Disorders related to excessive secretion of biologically active peptides by tumor cells include: fasting hypoglycemia due to increased insulin secretion from insulinoma-islet cell tumors; hypertension due to increased epinephrine and norepinephrine secreted from pheochromocytomas of the adrenal medulla and sympathetic paraganglia; and carcinoid syndrome, which includes abdominal cramps, diarrhea, and valvular heart disease, caused by excessive amounts of vasoactive substances (serotonin, bradykinin, histamine, prostaglandins, and polypeptide hormones) secreted from intestinal tumors. Ectopic synthesis and secretion of biologically active peptides (peptides not expected from a tumor) includes ACTH and vasopressin in lung and pancreatic cancers; parathyroid hormone in lung and bladder cancers; calcitonin in lung and breast cancers; and thyroid-stimulating hormone in medullary thyroid carcinoma.

Various human pathogens alter host cell protein trafficking pathways to their own advantage. For example, the HIV protein Nef downregulates cell-surface expression of CD4 molecules by accelerating their endocytosis through clathrin coated pits. This function of Nef is important for the spread of HIV from the infected cell (Harris, M. (1999) Curr. Biol. 9:R449-R461). A recently identified human protein, Nef-associated factor 1 (Naf1), a protein with four extended coiled-coil domains, has been found to associate with Nef. Overexpression of Naf1 increased cell surface expression of CD4, an effect which could be suppressed by Nef (Fukushi, M. et al. (1999) FEBS Lett. 442:83-88).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Genes Expressed in Breast Cancer

The potential application of gene expression profiling is relevant to improving diagnosis, prognosis, and treatment of disease. For example, both the levels and sequences expressed in tissues from subjects with breast cancer may be compared with the levels and sequences expressed in normal tissue.

There are more than 180,000 new cases of breast cancer diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Breast cancer is a genetic disease commonly caused by mutations in cellular disease. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to noninherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie et al., supra, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as BER-2/neu, BER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix Gla protein which is overexpressed is human breast carcinoma cells; Drgl or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou, Z. et al. (1998) Int. J. Cancer 78:95-99; Chen, L. et al. (1990) Oncogene 5:1391-1395; Uhix, W. et al (1999) FBBS Lett. 455:23-26; Sager, R. et al. (1996) CuiT. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Genes Expressed in Prostate Cancer

The potential application of gene expression profiling is also relevant to improving diagnosis, prognosis, and treatment of disease. For example, both the levels and sequences expressed in tissues from subjects with prostate cancer may be compared with the levels and sequences expressed in normal tissue.

Prostate cancer is a common malignancy in men over the age of 50, and the incidence increases with age. In the US, there are approximately 132,000 newly diagnosed cases of prostate cancer and more than 33,000 deaths from the disorder each year.

Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most cancers, prostate cancer develops through a multistage progression ultimately resulting in an aggressive, metastatic phenotype. The initial step in tumor progression involves the hyperproliferation of normal luminal and/or basal epithelial cells that become hyperplastic and evolve into early-stage tumors. The early-stage tumors are localized in the prostate but eventually may metastasize, particularly to the bone, brain or lung. About 80% of these tumors remain responsive to androgen treatment, an important hormone controlling the growth of prostate epithelial cells. However, in its most advanced state, cancer growth becomes androgen-independent and there is currently no known treatment for this condition.

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Current areas of cancer research provide additional prospects for markers as well as potential therapeutic targets for prostate cancer. Several growth factors have been shown to play a critical role in tumor development, growth, and progression. The growth factors Epidermal Growth Factor (EGF), Fibroblast Growth Factor (FGF), and Tumor Growth Factor alpha (TGFα) are important in the growth of normal as well as hyperproliferative prostate epithelial cells, particularly at early stages of tumor development and progression, and affect signaling pathways in these cells in various ways (Lin, J. et al. (1999) Cancer Res. 59:2891-2897; Putz, T. et al. (1999) Cancer Res. 59:227-233). The TGF-0 family of growth factors are generally expressed at increased levels in human cancers and the high expression levels in many cases correlates with advanced stages of malignancy and poor survival (Gold, L. I. (1999) Crit. Rev. Oncog. 10:303-360). Finally, there are human cell lines representing both the androgen-dependent stage of prostate cancer (LNCap) as well as the androgen-independent, hormone refractory stage of the disease (PC3 and DU145) that have proved useful in studying gene expression patterns associated with the progression of prostate cancer, and the effects of cell treatments on these expressed genes (Chung, T. D. (1999) Prostate 15:199-207).

Genes Expressed in Adipocyte Differentiation

The potential application of gene expression profiling is relevant to improving diagnosis, prognosis, and treatment of disease. For example, both the levels and sequences expressed in tissues from subjects with obesity or type II diabetes may be compared with the levels and sequences expressed in normal tissue.

The primary function of adipose tissue is the ability to store and release fat during periods of feeding and fasting. White adipose tissue is the major energy reserve in periods of fasting, and its reserve is mobilized during energy deprivation. Adipose tissue is one of the primary target tissues for insulin, and adipogenesis and insulin resistance are linked in type II diabetes, non-insulin dependent diabetes mellitus (NIDDM). Cytologically the conversion of a preadipocytes into mature adipocytes is characterized by deposition of fat droplets around the nuclei. The conversion process in vivo can be induced by thiazolidinediones (TZDs) and other PPARγ agonists (Adams, M. et al. (1997) J. Clin. Invest. 100:3149-3153) which also lead to increased sensitivity to insulin and reduced plasma glucose and blood pressure.

Thiazolidinediones (TZDs) act as agonists for the peroxisome-proliferator-activated receptor gamma (PPARγ), a member of the nuclear hormone receptor superfamily. TZDs reduce hyperglycemia, hyperinsulinemia, and hypertension, in part by promoting glucose metabolism and inhibiting gluconeogenesis. Roles for PPARγ and its agonists have been demonstrated in a wide range of pathological conditions including diabetes, obesity, hypertension, atherosclerosis, polycystic ovarian syndrome, and cancers such as breast, prostate, liposarcoma, and colon cancer.

The mechanism by which TZDs and other PPARγ agonists enhance insulin sensitivity is not fully understood, but may involve the ability of PPARγ to promote adipogenesis. When ectopically expressed in cultured preadipocytes, PPARγ is a potent inducer of adipocyte differentiation. TZDs, in combination with insulin and other factors, can also enhance differentiation of human preadipocytes in culture (Adams, et al., supra). The relative potency of different TZDs in promoting adipogenesis in vitro is proportional to both their insulin sensitizing effects in vivo, and their ability to bind and activate PPARγ in vitro. Interestingly, adipocytes derived from omental adipose depots are refractory to the effects of TZDs. It has therefore been suggested that the insulin sensitizing effects of TZDs may result from their ability to promote adipogenesis in subcutaneous adipose depots (Adams et al., supra). Further, dominant negative mutations in the PPARγ gene have been identified in two non-obese subjects with severe insulin resistance, hypertension, and overt non-insulin dependent diabetes mellitus (NEDDM) (Barroso, I. et al. (1998) Nature 402:880-883).

NIDDM is the most common form of diabetes mellitus, a chronic metabolic disease that affects 143 million people worldwide. NHDDM is characterized by abnormal glucose and lipid metabolism that result from a combination of peripheral insulin resistance and defective insulin secretion. NIDDM has a complex, progressive etiology and a high degree of heritability. Numerous complications of diabetes including heart disease, stroke, renal failure, retinopathy, and peripheral neuropathy contribute to the high rate of morbidity and mortality.

At the molecular level, PPARγ functions as a ligand activated transcription factor. In the presence of ligand, PPARγ forms a heterodimer with the retinoid X receptor (RXR) which then activates transcription of target genes containing one or more copies of a PPARγ response element (PPRE). Many genes important in lipid storage and metabolism contain PPREs and have been identified as PPARγ targets, including PEPCK, aP2, LPL, ACS, and FAT-P (Auwerx, J. (1999) Diabetologia 42:1033-1049). Multiple ligands for PPARγ have been identified. These include a variety of fatty acid metabolites; synthetic drugs belonging to the TZD class, such as Pioglitazone and Rosiglitazone (BRL49653); and certain non-glitazone tyrosine analogs such as G1262570 and GW1929. The prostaglandin derivative 15-dPGJ2 is a potent endogenous ligand for PPARγ.

Expression of PPARγ is very high in adipose but barely detectable in skeletal muscle, the primary site for insulin stimulated glucose disposal in the body. PPARγ is also moderately expressed in large intestine, kidney, liver, vascular smooth muscle, hematopoietic cells, and macrophages. The high expression of PPARγ in adipose suggests that the insulin sensitizing effects of TZDs may result from alterations in the expression of one or more PPARγ regulated genes in adipose tissue.

Identification of PPARγ target genes will contribute to better drug design and the development of novel therapeutic strategies for diabetes, obesity, and other conditions.

Systematic attempts to identify PPARγ target genes have been made in several rodent models of obesity and diabetes (Suzuki, A. et al. (2000) Jpn. J. Pharmacol. 84:113-123; Way, J. M. et al. (2001) Endocrinology 142:1269-1277). However, a serious drawback of the rodent gene expression studies is that significant differences exist between human and rodent models of adipogenesis, diabetes, and obesity (Taylor, S. I. (1999) Cell 97:9-12; Gregoire, F. M. et al. (1998) Physiol. Reviews 78:783-809). Therefore, an unbiased approach to identifying TZD regulated genes in primary cultures of human tissues is necessary to fully elucidate the molecular basis for diseases associated with PPARγ activity.

The majority of research in adipocyte biology to date has been done using transformed mouse preadipocyte cell lines. The culture condition, which stimulates mouse preadipocyte differentiation is different from that for inducing human primary preadipocyte differentiation. In addition, primary cells are diploid and may therefore reflect the in vivo context better than aneuploid cell lines. Understanding the gene expression profile during adipogenesis in human will lead to understanding the fundamental mechanism of adiposity regulation. Furthermore, through comparing the gene expression profiles of adipogenesis between donor with normal weight and donor with obesity, identification of crucial genes, potential drug targets for obesity and type H diabetes, will be possible.

There is a need in the art for new compositions, including nucleic acids and proteins, for the diagnosis, prevention, and treatment of vesicle trafficking disorders, autoimmune/inflammatory disorders, and cancer.

### SUMMARY OF THE INVENTION

Various embodiments of the invention provide purified polypeptides, vesicle-associated proteins, referred to collectively as ‘VAP’ and individually as ‘VAP-1,’ ‘VAP-2,’ ‘VAP-3,’ ‘VAP-4,’ ‘VAP-5,’ ‘VAP-6,’ ‘VAP-7,’ ‘VAP-8,’ ‘VAP-9,’ ‘VAP-10,’ ‘VAP-11,’ ‘VAP-12,’ ‘VAP-13,’ ‘VAP-14,’ ‘VAP-15,’ ‘VAP-16,’ ‘VAP-17,’ ‘VAP-18,’ ‘VAP-19,’ and ‘VAP-20’ and methods for using these proteins and their encoding polynucleotides for the detection, diagnosis, and treatment of diseases and medical conditions. Embodiments also provide methods for utilizing the purified vesicle-associated proteins and/or their encoding polynucleotides for facilitating the drug discovery process, including determination of efficacy, dosage, toxicity, and pharmacology. Related embodiments provide methods for utilizing the purified vesicle-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO: 1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides an isolated polypeptide comprising an amino acid sequence of SEQ ID NO:1-20.

Still another embodiment provides an isolated polynucleotide encoding a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. In another embodiment, the polynucleotide encodes a polypeptide selected from the group consisting of SEQ ID NO:1-20. In an alternative embodiment, the polynucleotide is selected from the group consisting of SEQ ID NO:21-40.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ NO: 1-20, and d) an immunogenic fragment of a polypeptide having an amino acid sequence selected from the group consisting of SEQ ID NO:1-20.

Still yet another embodiment provides an isolated polynucleotide selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20, and a pharmaceutically acceptable excipient. In one embodiment, the composition can comprise an amino acid sequence selected from the group consisting of SEQ ID NO:1-20. Other embodiments provide a method of treating a disease or condition associated with decreased or abnormal expression of functional VAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional VAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional VAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:21-40, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:21-40, ii) a polynucleotide comprising a naturally occurring polynucleotide sequence at least 90% identical or at least about 90% identical to a polynucleotide sequence selected from the group consisting of SEQ ID NO:21-40, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:21140, ii) a polynucleotide comprising a naturally occurring polynucleotide sequence at least 90% identical or at least about 90% identical to a polynucleotide sequence selected from the group consisting of SEQ ID NO:21-40, iji) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“VAP” refers to the amino acid sequences of substantially purified VAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of VAP. Agonists may include proteins, nucleic acids, carbohydrates, small molecules, or any other compound or composition which modulates the activity of VAP either by directly interacting with VAP or by acting on components of the biological pathway in which VAP participates.

An “allelic variant” is an alternative form of the gene encoding VAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding VAP include those sequences with deletions, insertions, or substitutions of different nucleotides, resulting in a polypeptide the same as VAP or a polypeptide with at least one functional characteristic of VAP. Included within this definition are polymorphisms which may or may not be readily detectable using a particular oligonucleotide probe of the polynucleotide encoding VAP, and improper or unexpected hybridization to allelic variants, with a locus other than the normal chromosomal locus for the polynucleotide encoding VAP. The encoded protein may also be “altered,” and may contain deletions, insertions, or substitutions of amino acid residues which produce a silent change and result in a functionally equivalent VAP. Deliberate amino acid substitutions may be made on the basis of one or more similarities in polarity, charge, solubility, hydrophobicity, hydrophilicity, and/or the amphipathic nature of the residues, as long as the biological or immunological activity of VAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of VAP. Antagonists may include proteins such as antibodies, anticalins, nucleic acids, carbohydrates, small molecules, or any other compound or composition which modulates the activity of VAP either by directly interacting with VAP or by acting on components of the biological pathway in which VAP participates.

The term “antibody” refers to intact immunoglobulin molecules as well as to fragments thereof, such as Fab, F(ab′)₂, and Fv fragments, which are capable of binding an epitopic determinant. Antibodies that bind VAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may beproduced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic VAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding VAP or fragments of VAP may be employed as hybridization probes. The probes may be stored in freezeried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of VAP or a polynucleotide encoding VAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:21-40 can comprise a region of unique polynucleotide sequence that specifically identifies SEQ ID NO:21-40, for example, as distinct from any other sequence in the genome from which the fragment was obtained. A fragment of SEQ ID NO:21-40 can be employed in one or more embodiments of methods of the invention, for example, in hybridization and amplification technologies and in analogous methods that distinguish SEQ ID NO:21-40 from related polynucleotides. The precise length of a fragment of SEQ ID NO:21-40 and the region of SEQ ID NO:21-40 to which the fragment corresponds are routinely determinable by one of ordinary skill in the art based on the intended purpose for the fragment.

A fragment of SEQ ID NO:1-20 is encoded by a fragment of SEQ ID NO:21-40. A fragment of SEQ ID NO:1-20 can comprise a region of unique amino acid sequence that specifically identifies SEQ ID NO:1-20. For example, a fragment of SEQ ID NO:1-20 can be used as an immunogenic peptide for the development of antibodies that specifically recognize SEQ ID NO:1-20. The precise length of a fragment of SEQ ID NO:1-20 and the region of SEQ ID NO:1-20 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLASTV. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (April-21-2000) set at default parameters. Such default parameters may be, for example:

Matrix: BLOSUM62

Rewardfor matchl: 1

Penalty for mismatch: −2

Open Gap: 5 and Extension Gap: 2 penalties

Gap×drop-off. 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (April-21-2000) with blastp set at default parameters. Such default parameters may be, for example:

Matrix: BLOSUM62

Open Gap: 11 and Extension Gap: 1 penalties

Gap×drop-off. 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., Cot or Rot analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of VAP which is capable of eliciting an immune response when introduced into a living organism, for example, a mammal. The term “immunogenic fragment” also includes any polypeptide or oligopeptide fragment of VAP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of VAP. For example, modulation may cause an increase or a decrease in protein activity, binding characteristics, or any other biological, functional, or immunological properties of VAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an VAP may involve lipidation, glycosylation, phosphorylation, acetylation, racemization, proteolytic cleavage, and other modifications known in the art. These processes may occur synthetically or biochemically. Biochemical modifications will ary by cell type depending on the enzymatic milieu of VAP.

“Probe” refers to nucleic acids encoding VAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MI Center for Genome Research, Cambridge Mass.) allows the user to input a “mispriming library,” in which sequences to avoid as primer binding sites are user-specified. Primer3 is useful, in particular, for the election of oligonucleotides for microarrays. (The source code for the latter two primer selection rograms may also be obtained from their respective sources and modified to meet the user's specific eeds.) The PrimeGen program (available to the public from the UK Human Genome Mapping roject Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing VAP, nucleic acids encoding VAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a niucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human vesicle-associated proteins (VAP), the polynucleotides encoding VAP, and the use of these compositions for the diagnosis, treatment, or prevention of vesicle trafficking disorders, autoimmune/inflammatory disorders, and cancer.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding licyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 sumnarize the properties of polypeptides of the invention, and these properties establish that the claimed polypeptides are vesicle-associated proteins. For example, SEQ ID NO:1 is 31% identical, from residue V2 to residue P272, to human Golgi membrane protein GP73 (GenBank ID g7271867) as determined by the Basic Local Alignment Search Tool (BLAST). (See Table 2.) The BLAST probability score is 9.4e-26, which indicates the probability of obtaining the observed polypeptide sequence alignment by chance. Data from TMHMMER analysis provides further corroborative evidence that SEQ ID NO:1 is a membrane protein localized to the Golgi apparatus. In another example, SEQ ID NO:3 is 99% identical, from residue M1 to residue D744, to human N-ethylraleimide-sensitive factor (GenBank ID g7920147) as determined by the Basic Local Alignment Search Tool (BLAST). (See Table 2.) The BLAST probability score is 0.0, which indicates the probability of obtaining the observed polypeptide sequence alignment by chance. SEQ ID NO:3 is localized to the subcellular region, has ATPase function, and has an AAA-protein family signature domain, as determined by BLAST analysis using the PROTEOME database. SEQ ID NO:3 also contains an ATPase family associated with various cellular activities (AAA) domain as determined by searching for statistically significant matches in the hidden Markov model (H)-based PFAM database of conserved protein family domains. (See Table 3.) Data from BLIMPS, MOTIFS, PROHESCAN, and additional BLAST analyses provide further corroborative evidence that SEQ ID NO:3 is a vesicular protein of the AAA family. In another example, SEQ ID NO:9 is 100% identical, from residue F2 to residue E92, to rat clathrin-associated protein 17 (GenBank ID g202928) as determined by the Basic Local Alignment Search Tool (BLAST). (See Table 2.) The BLAST probability score is 6.4E-45, which indicates the probability of obtaining the observed polypeptide sequence alignment by chance. SEQ ID NO:9 also has homology to human adaptor-related protein complex 2 sigma 1 subunit which is associated with clathrin coated vesicles and is involved in intracellular transport, as determined by BLAST analysis using the PROTEOME database. SEQ ID NO:9 also contains a clathrin adaptor complex small chain domain as determined by searching for statistically significant matches in the hidden Markov model (M)-based PFAM database of conserved protein family domains. (See Table 3.) Data from PROFILESCAN, MOTIFS, and additional BLAST analyses provide further corroborative evidence that SEQ ID NO:9 is a clathrin-associated protein. In another example, SEQ ID NO:10 is 95% identical, from residue M1 to residue M610, to rat clathrin assembly protein short form (GenBank ID g2792500) as determined by the Basic Local Alignment Search Tool (BLAST). (See Table 2.) The BLAST probability score is 0.0, which indicates the probability of obtaining the observed polypeptide sequence alignment by chance. As determined by BLAST analysis using the PROTEOME database, SEQ ID NO:10 also has homology to human and rat clathrin assembly lymphoid myeloid leukemia proteins which bind to clathrin heavy chain (CLTC) and play a role in coated pit internalization. Rearrangements in the corresponding lymphoid myeloid leukemia genes are associated with acute lymphoblastic and acute myeloid leukemias (PROTEOME IDs 2984951PICALM and 3335201Rn.10888). SEQ ID NO:10 also contains an ENTH (Epsin N-terminal homology) domain (a domain found in proteins involved in endocytosis and cytoskeletal machinery) as determined by searching for statistically significant matches in the hidden Markov model (HMM)-based PFAM database of conserved protein family domains. (See Table 3.) Data from additional BLAST and MOTIFS analyses provide further corroborative evidence that SEQ ID NO:10 is a clathrin assembly protein. In another example, SEQ ID NO:20 is 84% identical, from residue E17 to residue G262, to human syntaxin 4A (placental) (GenBank ID g12803245) as determined by the Basic Local Alignrment Search Tool (BLAST). (See Table 2.) The BLAST probability score is 2.6e-100, which indicates the probability of obtaining the observed polypeptide sequence alignment by chance. SEQ ID NO:20 also has homology to proteins that are localized to the cytoplasm, have SNAP receptor (t-SNARE) function, and are syntaxins, as determined by BLAST analysis using the PROTEOME database. SEQ ID NO:20 also contains a syntaxin domain, as determined by searching for statistically significant matches in the hidden Markov model (HMM)-based PFAM database of conserved protein family domains, and contains a SMRT t SNARE domain (helical region found in SNARES) and a SMRT_SynN domain as determined by searching for statistically significant matches in the hidden Markov model (HMM)-based SMRT database of conserved protein family domains. (See Table 3.) Data from BLIMPS, MOTIFS, and additional BLAST analyses provide further corroborative evidence that SEQ ID NO:20 is a syntaxin. SEQ ID NO:2, SEQ ID NO:4-8, and SEQ ID NO:11-19 were analyzed and annotated in a similar manner. The algorithms and parameters for the analysis of SEQ ID NO:1-20 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:21-40 or that distinguish between SEQ ID NO:21-40 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBB_{—}1_N is a “stretched” sequence, with XXXX being the Incyte project identification number, gAAAAA being the GenBank identification number of the human genomnic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the Genlank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, |
| ENST | for example, GENSCAN (Stanford University, |
| | CA, USA) or FGENES (Computer Genomics Group, |
| | The Sanger Centre, Cambridge, UK). |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location |
| | and EST composition data are combined to predict the |
| | exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP(SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses VAP variants. Various embodiments of VAP variants can have at least about 80%, at least about 90%, or at least about 95% amino acid sequence identity to the VAP amino acid sequence, and can contain at least one functional or structural characteristic of VAP.

Various embodiments also encompass polynucleotides which encode VAP. In a particular embodiment, the invention encompasses a polynucleotide sequence comprising a sequence selected from the group consisting of SEQ ID NO:21-40, which encodes VAP. The polynucleotide sequences of SEQ ID NO:21140, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding VAP. In particular, such a variant polynucleotide will have at least about 70%, or alternatively at least about 85%, or even at least about 95% polynucleotide sequence identity to a polynucleotide encoding VAP. A particular aspect of the invention encompasses a variant of a polynucleotide comprising a sequence selected from the group consisting of SEQ ID NO:21-40 which has at least about 70%, or alternatively at least about 85%, or even at least about 95% polynucleotide sequence identity to a nucleic acid sequence selected from the group consisting of SEQ ID NO:21-40. Any one of the polynucleotide variants described above can encode a polypeptide which contains at least one functional or structural characteristic of VAP.

In addition, or in the alternative, a polynucleotide variant of the invention is a splice variant of a polynucleotide encoding VAP. A splice variant may have portions which have significant sequence identity to a polynucleotide encoding VAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding VAP over its entire length; however, portions of the splice variant will have at least about 70%, or alternatively at least about 85%, or alternatively at least about 95%, or alternatively 100% polynucleotide sequence identity to portions of the polynucleotide encoding VAP. For example, a polynucleotide comprising a sequence of SEQ ID NO:30 and a polynucleotide comprising a sequence of SEQ ID NO:33 are splice variants of each other. Any one of the splice variants described above can encode a polypeptide which contains at least one functional or structural characteristic of VAP.

It will be appreciated by those skilled in the art that as a result of the degeneracy of the genetic code, a multitude of polynucleotide sequences encoding VAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring VAP, and all such variations are to be considered as being specifically disclosed.

Although polynucleotides which encode VAP and its variants are generally capable of hybridizing to polynucleotides encoding naturally occurring VAP under appropriately selected conditions of stringency, it may be advantageous to produce polynucleotides encoding VAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding VAP and its derivatives without altering the encoded amino acid sequences include the production of RNA transcripts having more desirable properties, such as a greater half-life, than transcripts produced from the naturally occurring sequence.

The invention also encompasses production of polynucleotides which encode VAP and VAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding VAP or any fragment thereof.

Embodiments of the invention can also include polynucleotides that are capable of hybridizing to the claimed polynucleotides, and, in particular, to those having the sequences shown in SEQ ID NO:21-40 and fragments thereof, under various conditions of stringency (Wahl, G. M. and S. L. Berger (1987) Methods Enzymol. 152:399407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding VAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1: 111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode VAP may be cloned in recombinant DNA molecules that direct expression of VAP, or fragments or functional equivalents thereof, in appropriate host cells. Due to the inherent degeneracy of the genetic code, other polynucleotides which encode substantially the same or a functionally equivalent polypeptides may be produced and used to express VAP.

The polynucleotides of the invention can be engineered using methods generally known in the art in order to alter VAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of VAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding VAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, VAP itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of VAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392421). The composition of the synthetic peptides may be confirmed by amino acid analysis or by sequencing (Creighton, supra, pp. 28-53).

In order to express a biologically active VAP, the polynucleotides encoding VAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding VAP. Such elements may vary in their strength and specificity. Specific initiation signals may also be used to achieve more efficient translation of polynucleotides encoding VAP. Such signals include the ATG initiation codon and adjacent sequences, e.g. the Kozak sequence. In cases where a polynucleotide sequence encoding VAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding VAP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding VAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding VAP. For example, routine cloning, subcloning, and propagation of polynucleotides encoding VAP can be achieved using a multifunctional E. coli vector such as PBLUESCRIPT (Stratagene, La Jolla Calif.) or PSPORT1 plasmid (Invitrogen). Ligation of polynucleotides encoding VAP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of VAP are needed, e.g. for the production of antibodies, vectors which direct high level expression of VAP may be used. For example, vectors containing the strong, inducible SP6 or 17 bacteriophage promoter may be used.

Yeast expression systems may be used for production of VAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of VAP. Transcription of polynucleotides encoding VAP may be driven by viral promoters, e.g., the ³⁵S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding VAP may be ligated into an adenovirus transcription/translation complex consisting of the late promoter and tripartite leader sequence. Insertion in a non-essential E1 or E3 region of the viral genome may be used to obtain infective virus which expresses VAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in manmmalian systems, stable expression of VAP in cell lines is preferred. For example, polynucleotides encoding VAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr^{·} cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dlfr confers resistance to methotrexate; neo confers resistance to the aminoglycosides neomycin and GA418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding VAP is inserted within a marker gene sequence, transformed cells containing polynucleotides encoding VAP can be identified by the absence of marker gene function. Alternatively, a marker gene can be placed in tandem with a sequence encoding VAP under the control of a single promoter. Expression of the marker gene in response to induction or selection usually indicates expression of the tandem gene as well.

In general, host cells that contain the polynucleotide encoding VAP and that express VAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of VAP using either specific polyclonal or monoclonal antibodies are known in the art. Examples of such techniques include enzymeinked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on VAP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding VAP include oligolabeling, nick translation, end-labeling, or PCR amplification using a labeled nucleotide. Alternatively, polynucleotides encoding VAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison W), and US Biochemical. Suitable reporter molecules or labels which may be used for ease of detection include radionuclides, enzymes, fluorescent, cherniluminescent, or chromogenic agents, as well as substrates, cofactors, inhibitors, magnetic particles, and the like.

Host cells transformed with polynucleotides encoding VAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode VAP may be designed to contain signal sequences which direct secretion of VAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HBEK293, and W138) are available from the American Type Culture Collection (ATCC, Manassas Va.) and may be chosen to ensure the correct modification and processing of the foreign protein.

In another embodiment of the invention, natural, modified, or recombinant polynucleotides encoding VAP may be ligated to a heterologous sequence resulting in translation of a fusion protein in any of the aforementioned host systems. For example, a chimeric VAP protein containing a heterologous moiety that can be recognized by a commercially available antibody may facilitate the screening of peptide libraries for inhibitors of VAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-nzyc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the VAP encoding sequence and the heterologous protein sequence, so that VAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled VAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

VAP, fragments of VAP, or variants of VAP may be used to screen for compounds that specifically bind to VAP. One or more test compounds may be screened for specific binding to VAP. In various embodiments, 1, 2, 3, 4, 5, 10, 20, 50, 100, or 200 test compounds can be screened for specific binding to VAP. Examples of test compounds can include antibodies, anticalins, oligonucleotides, proteins (e.g., ligands or receptors), or small molecules.

In related embodiments, variants of VAP can be used to screen for binding of test compounds, such as antibodies, to VAP, a variant of VAP, or a combination of VAP and/or one or more variants VAP. In an embodiment, a variant of VAP can be used to screen for compounds that bind to a variant of VAP, but not to VAP having the exact sequence of a sequence of SEQ ID NO:1-20. VAP variants used to perform such screening can have a range of about 50% to about 99% sequence identity to VAP, with various embodiments having 60%, 70%, 75%, 80%, 85%, 90%, and 95% sequence identity.

In an embodiment, a compound identified in a screen for specific binding to VAP can be closely related to the natural ligand of VAP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor VAP (Howard, A. D. et al. (2001) Trends Pharmacol. Sci. 22: 132-140; Wise, A. et al. (2002) Drug Discovery Today 7:235-246).

In other embodiments, a compound identified in a screen for specific binding to VAP can be losely related to the natural receptor to which VAP binds, at least a fragment of the receptor, or a ragment of the receptor including all or a portion of the ligand binding site or binding pocket. For example, the compound may be a receptor for VAP which is capable of propagating a signal, or a decoy receptor for VAP which is not capable of propagating a signal (Ashkenazi, A. and V. M. Divit (1999) Curr. Opin. Cell Biol. 11:255-260; Mantovani, A. et al. (2001) Trends Immunol. 22:328-336).

The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG1 (Taylor, P. C. et al. (2001) Curr. Opin. Immunol. 13:611-616).

In one embodiment, two or more antibodies having similar or, alternatively, different specificities can be screened for specific binding to VAP, fragments of VAP, or variants of VAP. The binding specificity of the antibodies thus screened can thereby be selected to identify particular fragments or variants of VAP. In one embodiment, an antibody can be selected such that its binding specificity allows for preferential identification of specific fragments or variants of VAP. In another embodiment, an antibody can be selected such that its binding specificity allows for preferential diagnosis of a specific disease or condition having increased, decreased, or otherwise abnormal production of VAP.

In an embodiment, anticalins can be screened for specific binding to VAP, fragments of VAP, or variants of VAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit VAP involves producing appropriate cells which express VAP, either as a secreted protein or on the cell membrane. Preferred cells can include cells from mammals, yeast, Drosophila, or E. coli. Cells expressing VAP or cell membrane fractions which contain VAP are then contacted with a test compound and binding, stimulation, or inhibition of activity of either VAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with VAP, either in solution or affixed to a solid support, and detecting the binding of VAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

VAP, fragments of VAP, or variants of VAP may be used to screen for compounds that modulate the activity of VAP. Such compounds may include agonists, antagonists, or partial or inverse agonists. In one embodiment, an assay is performed under conditions permissive for VAP activity, wherein VAP is combined with at least one test compound, and the activity of VAP in the presence of a test compound is compared with the activity of VAP in the absence of the test compound. A change in the activity of VAP in the presence of the test compound is indicative of a compound that modulates the activity of VAP. Alternatively, a test compound is combined with an in vitro or cell-free system comprising VAP under conditions suitable for VAP activity, and the assay is performed. In either of these assays, a test compound which modulates the activity of VAP may do so indirectly and need not come in direct contact with the test compound. At least one and up to a plurality of test compounds may be screened.

In another embodiment, polynucleotides encoding VAP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding VAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding VAP can also be used to create “knockin” humanized animals (pigs) or transgenic animals (mice or rats) to model human disease. With knockin technology, a region of a polynucleotide encoding VAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress VAP, e.g., by secreting VAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of VAP and vesicle-associated proteins. Expression of VAP is closely associated with lung tissue, ovary tissue, prostatic tumor tissue, adipocyte tissue, metastatic bone marrow neuroblastoma tissue, brain tissue, colon tissue, testiular tissue, and muscle tissue. In addition, examples of tissues expressing VAP can be found in Table 6 and can also be found in Example XI. Therefore, VAP appears to play a role in vesicle trafficking disorders, autoimmune/inflammatory disorders, and cancer. In the treatment of disorders associated with increased VAP expression or activity, it is desirable to decrease the expression or activity of VAP. In the treatment of disorders associated with decreased VAP expression or activity, it is desirable to increase the expression or activity of VAP.

Therefore, in one embodiment, VAP or a fragment or derivative thereof may be administered to a subject to treat or prevent a disorder associated with decreased expression or activity of VAP. Examples of such disorders include, but are not limited to, a vesicle trafficking disorder, such as cystic fibrosis, glucose-galactose malabsorption syndrome, hypercholesterolemia, diabetes mellitus, diabetes insipidus, hyper- and hypoglycemia, Grave's disease, goiter, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, myasthenia gravis, rheumatoid and osteoarthritis, scleroderma, Chediak-Higashi and Sjogren's syndromes, systemic lupus erythematosus, toxic shock syndrome, traumatic tissue damage, and viral, bacterial, fungal, helminthic, and protozoal infections; an autoimmune/inflammatory disorder, such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing VAP or a fragment or derivative thereof may be administered to a subject to treat or prevent a disorder associated with decreased expression or activity of VAP including, but not limited to, those described above.

In a further embodiment, a composition comprising a substantially purified VAP in conjunction with a suitable pharmaceutical carrier may be administered to a subject to treat or prevent a disorder associated with decreased expression or activity of VAP including, but not limited to, those provided above.

In still another embodiment, an agonist which modulates the activity of VAP may be administered to a subject to treat or prevent a disorder associated with decreased expression or activity of VAP including, but not limited to, those listed above.

In a further embodiment, an antagonist of VAP may be administered to a subject to treat or prevent a disorder associated with increased expression or activity of VAP. Examples of such disorders include, but are not limited to, those vesicle trafficking disorders, autoimmune/inflammatory disorders, and cancer described above. In one aspect, an antibody which specifically binds VAP may be used directly as an antagonist or indirectly as a targeting or delivery mechanism for bringing a pharmaceutical agent to cells or tissues which express VAP.

In an additional embodiment, a vector expressing the complement of the polynucleotide encoding VAP may be administered to a subject to treat or prevent a disorder associated with increased expression or activity of VAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of VAP may be produced using methods which are generally known in the art. In particular, purified VAP may be used to produce antibodies or to screen libraries of pharmaceutical agents to identify those which specifically bind VAP. Antibodies to VAP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with VAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to VAP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of VAP amino acids may be fused with those of another protein, such as KLH, and antibodies to the chimeric molecule may be produced.

Monoclonal antibodies to VAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natd. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce VAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for VAP may also be generated. For example, such fragments include, but are not limited to, F(ab′)₂ fragments produced by pepsin digestion of the antibody molecule and Fab fragments generated by reducing the disulfide bridges of the F(ab)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between VAP and its specific antibody. A two-site, monoclonal-based immunoassay utilizing monoclonal antibodies reactive to two non-interfering VAP epitopes is generally used, but a competitive binding assay may also be employed (Pound, supra).

Various methods such as Scatchard analysis in conjunction with radioimmunoassay techniques may be used to assess the affinity of antibodies for VAP. Affinity is expressed as an association constant, Kₐ, which is defined as the molar concentration of VAP-antibody complex divided by the molar concentrations of free antigen and free antibody under equilibrium conditions. The Kₐ determined for a preparation of polyclonal antibodies, which are heterogeneous in their affinities for multiple VAP epitopes, represents the average affinity, or avidity, of the antibodies for VAP. The Kₐ determined for a preparation of monoclonal antibodies, which are monospecific for a particular VAP epitope, represents a true measure of affinity. High-affinity antibody preparations with Kₐ ranging from about 10⁹ to 10¹² L/mole are preferred for use in immunoassays in which the VAP-antibody complex must withstand rigorous manipulations. Low-affinity antibody preparations with Kₐ ranging from about 106 to 107 L/mole are preferred for use in immunopurification and similar procedures which ultimately require dissociation of VAP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of VAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding VAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding VAP. Such technology is well known in the art, and antisense oligonucleotides or larger fragments can be designed from various locations along the coding or control regions of sequences encoding VAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding VAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VR1 or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosonia cruzi). In the case where a genetic deficiency in VAP expression or regulation causes disease, the expression of VAP from an appropriate population of transduced cells may alleviate the clinical manifestations caused by the genetic deficiency.

In a further embodiment of the invention, diseases or disorders caused by deficiencies in VAP are treated by constructing mammalian expression vectors encoding VAP and introducing these vectors by mechanical means into VAP-deficient cells. Mechanical transfer technologies for use with cells iii vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Recipon (1998) Curr. Opin. Biotechnol. 9:445-450).

Expression vectors that may be effective for the expression of VAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). VAP may be expressed using (i) a constitutively active promoter, (e.g., from cytomegalovirus (CMV), Rous sarcoma virus (RSV), SV40 virus, thymidine kinase (TK), or P-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding VAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to VAP expression are treated by constructing a retrovirus vector consisting of (i) the polynucleotide encoding VAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:47074716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding VAP to cells which have one or more genetic abnormalities with respect to the expression of VAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding VAP to target cells which have one or more genetic abnormalities with respect to the expression of VAP. The use of herpes simplex virus (HSV)-based vectors may be especially valuable for introducing VAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding VAP to target cells. The biology of the prototypic alphavirus, Sernliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normnally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for VAP into the alphavirus genome in place of the capsid-coding region results in the production of a large number of VAP-coding RNAs and the synthesis of high levels of VAP in vector transduced cells. While alphavirus infection is typically associated with cell lysis within a few days, the ability to establish a persistent infection in harnster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of VAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding VAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding VAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAI) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in marnmalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brurnmelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding VAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased VAP expression or activity, a compound which specifically inhibits expression of the polynucleotide encoding VAP may be therapeutically useful, and in the treatment of disorders associated with decreased VAP expression or activity, a compound which specifically promotes expression of the polynucleotide encoding VAP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding VAP is exposed to at least one test compound thus obtained. The sample may comprise, for example, an intact or permeabilized cell, or an in vitro cell-free or reconstituted biochemical system. Alterations in the expression of a polynucleotide encoding VAP are assayed by any method commonly known in the art. Typically, the expression of a specific nucleotide is detected by hybridization with a probe having a nucleotide sequence complementary to the sequence of the polynucleotide encoding VAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of VAP, antibodies to VAP, and mimetics, agonists, antagonists, or inhibitors of VAP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising VAP or fragments thereof. For example, liposome preparations containing a cell-impermeable macromolecule may promote cell fusion and intracellular delivery of the macromolecule. Alternatively, VAP or a fragment thereof may be joined to a short cationic N-terminal portion from the HEV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example VAP or fragments thereof, antibodies of VAP, and agonists, antagonists or inhibitors of VAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind VAP may be used for the diagnosis of disorders characterized by expression of VAP, or in assays to monitor patients being treated with VAP or agonists, antagonists, or inhibitors of VAP. Antibodies useful for diagnostic purposes may be prepared in the same manner as described above for therapeutics. Diagnostic assays for VAP include methods which utilize the antibody and a label to detect VAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring VAP, including ELISAs, RIAs, and FACS, are known in the art and provide a basis for diagnosing altered or abnormal levels of VAP expression. Normal or standard values for VAP expression are established by combining body fluids or cell extracts taken from normal mammalian subjects, for example, human subjects, with antibodies to VAP under conditions suitable for complex formation. The amount of standard complex formation may be quantitated by various methods, such as photometric means. Quantities of VAP expressed in subject, control, and disease samples from biopsied tissues are compared with the standard values. Deviation between standard and subject values establishes the parameters for diagnosing disease.

In another embodiment of the invention, polynucleotides encoding VAP may be used for diagnostic purposes. The polynucleotides which may be used include oligonucleotides, complementary RNA and DNA molecules, and PNAs. The polynucleotides may be used to detect and quantify gene expression in biopsied tissues in which expression of VAP may be correlated with disease. The diagnostic assay may be used to determine absence, presence, and excess expression of VAP, and to monitor regulation of VAP levels during therapeutic intervention.

In one aspect, hybridization with PCR probes which are capable of detecting polynucleotides, including genomic sequences, encoding VAP or closely related molecules may be used to identify nucleic acid sequences which encode VAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding VAP, allelic variants, or related sequences.

Probes may also be used for the detection of related sequences, and may have at least 50% sequence identity to any of the VAP encoding sequences. The hybridization probes of the subject invention may be DNA or RNA and may be derived from the sequence of SEQ ID NO:21-40 or from genomic sequences including promoters, enhancers, and introns of the VAP gene.

Means for producing specific hybridization probes for polynucleotides encoding VAP include the cloning of polynucleotides encoding VAP or VAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidinhbiotin coupling systems, and the like.

Polynucleotides encoding VAP may be used for the diagnosis of disorders associated with expression of VAP. Examples of such disorders include, but are not limited to, a vesicle trafficking disorder, such as cystic fibrosis, glucose-galactose malabsorption syndrome, hypercholesterolemia, diabetes mellitus, diabetes insipidus, hyper- and hypoglycemia, Grave's disease, goiter, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, myasthenia gravis, rheumatoid and osteoarthritis, scleroderma, Chediak-Higashi and Sjogren's syndromes, systemic lupus erythematosus, toxic shock syndrome, traumatic tissue damage, and viral, bacterial, fungal, helminthic, and protozoal infections, an autoimmune/inflammatory disorder, such as acquired immunodeficiency syndrome (AIDS), Addison's disease, adult respiratory distress syndrome, allergies, ankylosing spondylitis, amyloidosis, anemia, asthma, atherosclerosis, autoinmmune hemolytic anemia, autoimmune thyroiditis, autoimmune polyendocrinopathy-candidiasis-ectodermal dystrophy (APECED), bronchitis, cholecystitis, contact ermatitis, Crohn's disease, atopic dermatitis, dermatomyositis, diabetes mellitus, emphysema, pisodic lymphopenia with lymphocytotoxins, erythroblastosis fetalis, erythema nodosum, atrophic astritis, glomerulonephritis, Goodpasture's syndrome, gout, Graves' disease, Hashimoto's hyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helninthic infections, and trauma; and a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Polynucleotides encoding VAP may be used in Southern or northern analysis, dot blot, or other membrane-based technologies; in PCR technologies; in dipstick, pin, and multiformat ELISA-like assays; and in microarrays utilizing fluids or tissues from patients to detect altered VAP expression. Such qualitative or quantitative methods are well known in the art.

In a particular embodiment, polynucleotides encoding VAP may be used in assays that detect the presence of associated disorders, particularly those mentioned above. Polynucleotides complementary to sequences encoding VAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding VAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of VAP, a normal or standard profile for expression is established. This may be accomplished by combining body fluids or cell extracts taken from normal subjects, either animal or human, with a sequence, or a fragment thereof, encoding VAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding VAP may involve the use of PCR. These oligomers may be chemically synthesized, generated enzymatically, or produced in vitro. Oligomers will preferably contain a fragment of a polynucleotide encoding VAP, or a fragment of a polynucleotide complementary to the polynucleotide encoding VAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding VAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding VAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of VAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, VAP, fragments of VAP, or antibodies specific for VAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for VAP to quantify the levels of VAP expression. In one embodiment, the antibodies are used as elements on a microarray, and protein expression levels are quantified by exposing the microarray to the sample and detecting the levels of protein bound to each array element (Lueling, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweileret al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding VAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to, a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding VAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 1 lq22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, VAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between VAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with VAP, or fragments thereof, and washed. Bound VAP is then detected by methods well known in the art. Purified VAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding VAP specifically compete with a test compound for binding VAP. In this manner, antibodies can be used to detect the presence of any peptide which shares one or more antigenic determinants with VAP.

In additional embodiments, the nucleotide sequences which encode VAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. Thefollowing embodiments are, therefore, to be construed as merely illustrative, and not limitative of the remainder of the disclosure in any way whatsoever.

The disclosures of all patents, applications, and publications mentioned above and below, including U.S. Ser. No. 60/347,927, U.S. Ser. No. 60/332,908, U.S. Ser. No. 60/331,865, U.S. Ser. No. 60/342,604, and U.S. Ser. No. 60/354,827, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIAP vector system (Stratagene) or by cell lysis. Plasmids were purified using at least one of the following: a Magic or WIARD Minipreps DNA purification system (Promega); an AGTC Miniprep purification kit (Edge Biosystems, Gaithersburg Md.); and QIAWELL 8 Plasmid, QIAWELL 8 Plus Plasmnid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 84-well plates, and the concentration of amplified plasmid DNA was quantified fluorometrically sing PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch.

7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic progranmning, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:4143); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HI is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MBGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:21-40. Fragments from about 20 to about 4000 nucleotides which are useful in hybridization and amplification technologies are described in Table 4, column 2.

IV. Identification and Editing of Coding Sequences from Genomic DNA

Putative vesicle-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for enscan to analyze at once was set to 30 kb. To determine which of these Genscan predicted cDNA equences encode vesicle-associated proteins, the encoded polypeptides were analyzed by querying against PFAM models for vesicle-associated proteins. Potential vesicle-associated proteins were also identified by homology to Incyte cDNA sequences that had been annotated as vesicle-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomnic Sequence Data with cDNA Sequence Data “Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of VAP Encoding Polynucleotides

The sequences which were used to assemble SEQ ID NO:21-40 were compared with sequences from the Incyte LIFESEQ database and public domain databases using BLAST and other implementations of the Smith-Waterman algorithm. Sequences from these databases that matched SEQ ID NO:21-40 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centimorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding VAP are analyzed with respect to the tissue sources from which they were derived. For example, some full length sequences are assembled, at least in part, with overlapping Incyte cDNA sequences (see Example E). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding VAP. cDNA sequences and cDNA library/tissue information are found in the LIFESEQ GOLD database (Incyte Genomics, Palo Alto Calif.).

VIII. Extension of VAP Encoding Polynucleotides

Full length polynucleotides are produced by extension of an appropriate fragment of the full. length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Me²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in VAP Encoding Polynucleotides

Common DNA sequence variants known as single nucleotide polymorphisms (SNPs) were identified in SEQ ID NO:21-40 using the LIFESEQ database (Incyte Genomics). Sequences from the same gene were clustered together and assembled as described in Example m, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:21-40 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 107 counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 μg/μl oligo-(dT) primer (21 mer), 1×first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-CyS (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of CyS. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

For example, SEQ ID NO:30 and SEQ ID NO:33 showed differential expression in certain breast carcinoma cell lines versus primary mammary epithelial cells as determined by microarray analysis. The gene expression profile of a primary mammary epithelial cell line, HMEC, was compared to the gene expression profiles of breast carcinoma lines at different stages of tumor progression. Cell lines compared included: a) MCF7, a nonmalignant breast adenocarcinoma cell line isolated from the pleural effusion of a 69-year-old female; b) T47D, a breast carcinoma cell line isolated from a pleural effusion obtained from a 54-year-old female with an infiltrating ductal carcinoma of the breast; c) Sk-BR-3, a breast adenocarcinoma cell line isolated from a malignant pleural effusion of a 43-year-old female; d) BT-20, a breast carcinoma cell line derived int vitro from tumor mass isolated from a 74-year-old female; e) MDA-mb-435S, a spindle shaped strain that evolved from the parent line (435) isolated from the pleural effusion of a 31-year-old female with metastatic, ductal adenocarcinoma of the breast; and f) MDA-mb-231, a metastatic breast tumor cell line derived from the pleural effusion of a 51-year-old female with metastatic breast carcinoma. The microarray experiments showed that the expression of SEQ ID NO:30 and SEQ ID NO:33 were decreased by at least two fold in cells from carcinoma cell lines (MCF7, Sk-BR-3, and T47D) relative to cells from the primary mammary epithelial cell line, HMEC. Therefore, in various embodiments, SEQ ID NO:30 and SEQ ID NO:33 can be used for one or more of the following: i) monitoring treatment of breast cancer, ii) diagnostic assays for breast cancer, and iii) developing therapeutics and/or other treatments for breast cancer.

Furthermore, the expression of SEQ ID NO:30 and SEQ ID NO:33 were decreased by at least two-fold in treated human adipocytes from obese and normal donors when compared to non-treated adipocytes from the same donors. The normal human primary subcutaneous preadipocytes were isolated from adipose tissue of a 28-year-old healthy female with a body mass index (BMI) of 23.59.

The obese human primary subcutaneous preadipocytes were isolated from adipose tissue of a 40-year-old healthy female with a body mass index (BMI) of 32.47. The preadipocytes were cultured and induced to differentiate into adipocytes by culturing them in the differentiation medium containing the active components, PPAR-γ agonist and human insulin. Human preadipocytes were treated with human insulin and PPAR-γ agonist for three days and subsequently were switched to medium containing insulin for 24 hours, 48 hours, four days, 8 days or 15 days before the cells were collected for analysis. Differentiated adipocytes were compared to untreated preadipocytes maintained in culture in the absence of inducing agents. Between 80% and 90% of the preadipocytes finally differentiated to adipocytes as observed under phase contrast microscope. Therefore, in various embodiments, SEQ ID NO:30 and SEQ ID NO:33 can be used for one or more of the following: i) monitoring treatment of diabetes mellitus and other disorders, such as obesity, hypertension, and atherosclerosis, ii) diagnostic assays for diabetes mellitus and other disorders, such as obesity, hypertension, and atherosclerosis, and iii) developing therapeutics and/or other treatments for diabetes mellitus and other disorders, such as obesity, hypertension, and atherosclerosis.

In yet another example, SEQ ID NO:30 showed differential expression in the PC3 prostate carcinoma cell line versus normal prostate epithelial cells as determined by microarray analysis. Three prostate carcinoma cell lines, DU145, LNCAP, and PC-3 were included in the experiments. DU145 was isolated from a metastatic site in the brain of a 69-year-old male with widespread metastatic prostate carcinoma. DU145 has no detectable sensitivity to hormones; forms colonies in semi-solid medium; is only weekly positive for acid phosphatase; and cells are negative for prostate specific antigen (PSA). LNCaP is a prostate carcinoma cell line isolated from a lymph node biopsy of a 50-year-old male with metastatic prostate carcinoma. LNCaP expresses PSA, produces prostate acid phosphatase, and expresses androgen receptors. PC-3, a prostate adenocarcinoma cell line, was isolated from a metastatic site in the bone of a 62-year-old male with grade IV prostate adenocarcinoma. The normal epithelial cell line, PrEC, is a primary prostate epithelial cell line isolated from a normal donor. In one experiment, the expression of cDNAs from the prostate carcinoma cell lines representing various stages of prostate tumor progression were compared with that of the normal prostate epithelial cells under the same culture conditions. The result from this experiment showed that the expression of SEQ ID NO:30 was decreased by at least two fold in PC3 cells compared to PrEC cells. In a separate experiment, the expression of cDNAs from the prostate carcinoma cell lines grown under optimal conditions (in the presence of growth factors and nutrients) were compared to that of the normal prostate epithelial cells grown under restrictive conditions (in the absence of growth factors and hormones). This experiment showed that the expression of SEQ ID NO:30 was decreased by at least two fold in PC-3 prostate carcinoma lines grown under optimal conditions relative to PrECs grown under restrictive conditions. Therefore, in various embodiments, SEQ ID NO:30 can be used for one or more of the following: i) monitoring treatment of prostate cancer, ii) diagnostic assays for prostate cancer, and iii) developing therapeutics and/or other treatments for prostate cancer.

In another example, SEQ ID NO:40 was differentially expressed in treated as compared to untreated human THP-1 cells. THP-1 cells are a promonocyte cell line isolated from the peripheral blood of a 1-year-old male with acute monocytic leukemia. Upon stimulation with PMA, THP-1 differentiates into macrophage-like cells that display many characteristics of peripheral human macrophages. THP-1 cells have been extensively used in the study of signaling in human monocytes and the identification of new factors produced by human monocytes. PMA activator is a broad activator of the protein kinase C-dependent pathways. Ionomycin is a calcium-ionophore that permits the entry of calcium in the cell, thus increasing the cytosolic calcium concentration. The combination of PMA and ionomycin activates two of the major signaling pathways used by mammalian cells to interact with their environment. In T cells, the combination of PMA and ionomycin mimics the type of secondary signaling events elicited during optimal B cell activation.

THP-1 cells were stimulated in vitro with soluble PMA and ionomycin for 0.5, 1, 2, 4, and 8 hours. The treated cells were compared to untreated THP-1 cells kept in culture in the absence of stimuli. SEQ ID NO:40 was overexpressed by at least two-fold in THP-1 cells treated for 2, 4, and 8 hours as compared to untreated counterparts. Therefore, in various embodiments, SEQ ID NO:40 can be used for one or more of the following: i) monitoring treatment of autoimmune/inflammatory disorders, ii) diagnostic assays for autoimmune/inflammatory disorders, and iii) developing therapeutics and/or other treatments for autoimmune/inflammatory disorders.

XI. Complementary Polynucleotides

Sequences complementary to the VAP-encoding sequences, or any parts thereof, are used to detect, decrease, or inhibit expression of naturally occurring VAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of VAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the VAP-encoding transcript.

XIII. Expression of VAP

Expression and purification of VAP is achieved using bacterial or virus-based expression systems. For expression of VAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express VAP upon induction with isopropyl beta-D-thiogalactopyranoside (IPTG). Expression of VAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding VAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, VAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from VAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified VAP obtained by these methods can be used directly in the assays shown in Examples XVII and xvm, where applicable.

XIV. Functional Assays

VAP function is assessed by expressing the sequences encoding VAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of VAP on gene expression can be assessed using highly purified populations of cells transfected with sequences encoding VAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding VAP and other genes of interest can be analyzed by northern analysis or microarray techniques.

XV. Production of VAP Specific Antibodies

VAP substantially purified using polyacrylamide gel electrophoresis (PAGE; see, e.g., Harrington, M. G. (1990) Methods Enzymol. 182:488495), or other purification techniques, is used to immunize animals (e.g., rabbits, mice, etc.) and to produce antibodies using standard protocols.

Alternatively, the VAP amino acid sequenceis analyzed using LASERGENE software (DNASTAR) to determnine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosysters) using FMOC chemistry and coupled to KLH (Sigrna-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-VAP activity by, for example, binding the peptide or VAP to a substrate, blocking with 1% BSA, reacting with rabbit antisera, washing, and reacting with radio-iodinated goat anti-rabbit IgG.

XVI. Purification of Naturally Occurring VAP Using Specific Antibodies

Naturally occurring or recombinant VAP is substantially purified by immunoaffinity chromatography using antibodies specific for VAP. An immunoaffinity column is constructed by covalently coupling anti-VAP antibody to an activated chromatographic resin, such as CNBr-activated SEPHAROSE (Amersham Biosciences). After the coupling, the resin is blocked and washed according to the manufacturer's instructions.

Media containing VAP are passed over the immunoaffinity column, and the column is washed under conditions that allow the preferential absorbance of VAP (e.g., high ionic strength buffers in the presence of detergent). The column is eluted under conditions that disrupt antibody/VAP binding (e.g., a buffer of pH 2 to pH 3, or a high concentration of a chaotrope, such as urea or thiocyanate ion), and VAP is collected.

XVII. Identification of Molecules Which Interact with VAP

VAP, or biologically active fragments thereof, are labeled with ¹²⁵I Bolton-Hunter reagent (Bolton, A. E. and W. M. Hunter (1973) Biocheim J. 133:529-539). Candidate molecules previously arrayed in the wells of a multi-well plate are incubated with the labeled VAP, washed, and any wells with labeled VAP complex are assayed. Data obtained using different concentrations of VAP are used to calculate values for the number, affinity, and association of VAP with the candidate molecules.

Alternatively, molecules interacting with VAP are analyzed using the yeast two-hybrid system as described in Fields, S. and O. Song (1989; Nature 340:245-246), or using commercially available kits based on the two-hybrid system, such as the MATCHMAKER system (Clontech).

VAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of VAP Activity

VAP activity is measured by its inclusion in coated vesicles. VAP can be expressed by transforming a mammalian cell line such as COS7, HeLa, or CHO with an eukaryotic expression vector encoding VAP. Eukaryotic expression vectors are commercially available, and the techniques to introduce them into cells are well known to those skilled in the art. A small amount of a second plasmid, which expresses any one of a number of marker genes, such as O-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of VAP and p-galactosidase.

Transformed cells are collected and cell lysates are assayed for vesicle formation. A non-hydrolyzable form of GTP, GTPγS, and an ATP regenerating system are added to the lysate and the mixture is incubated at 37° C. for 10 minutes. Under these conditions, over 90% of the vesicles remain coated (Orci, L. et al (1989) Cell 56:357-368). Transport vesicles are salt-released from the Golgi membranes, loaded under a sucrose gradient, centrifuged, and fractions are collected and analyzed by SDS-PAGE. Co-localization of VAP with clathrin or COP coatamer is indicative of VAP activity in vesicle formation. The contribution of VAP to vesicle formation can be confirmed by incubating lysates with antibodies specific for VAP prior to GTPγS addition. The antibody will bind to VAP and interfere with its activity, thus preventing vesicle formation.

In the alternative, VAP activity is measured by its ability to alter vesicle trafficking pathways. Vesicle trafficking in cells transformed with VAP is examined using fluorescence microscopy. Antibodies specific for vesicle coat proteins or typical vesicle trafficking substrates such as transferrin or the mannose-6-phosphate receptor are commercially available. Various cellular components such as ER, Golgi bodies, peroxisomes, endosomes, lysosomes, and the plasmalemma are examined. Alterations in the numbers and locations of vesicles in cells transformed with VAP as compared to control cells are characteristic of VAP activity.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | Incyte | | Incyte | Incyte |
|---|---|---|---|---|---|
| Incyte | Polypeptide | Polypeptide | Polynucleotide | Polynucleotide | Full Length |
| Project ID | SEQ ID NO: | ID | SEQ ID NO: | ID | Clones |
| 7500521 | 1 | 7500521CD1 | 21 | 7500521CB1 | 6369107CA2, |
| | | | | | 90033542CA2, |
| | | | | | 90116770CA2, |
| | | | | | 90118910CA2, |
| | | | | | 90119020CA2, |
| | | | | | 90119081CA2, |
| | | | | | 90119090CA2, |
| | | | | | 90119101CA2, |
| | | | | | 90119173CA2, |
| | | | | | 90119202CA2, |
| | | | | | 90119257CA2, |
| | | | | | 90119265CA2, |
| | | | | | 90119273CA2, |
| | | | | | 90119289CA2 |
| 7502992 | 2 | 7502992CD1 | 22 | 7502992CB1 | 90174555CA2, |
| | | | | | 90174563CA2, |
| | | | | | 90174571CA2, |
| | | | | | 90174579CA2, |
| | | | | | 90174587CA2, |
| | | | | | 90174679CA2, |
| | | | | | 90174695CA2 |
| 71187173 | 3 | 71187173CD1 | 23 | 71187173CB1 | 2159469CA2 |
| 7503143 | 4 | 7503143CD1 | 24 | 7503143CB1 | |
| 7503563 | 5 | 7503563CD1 | 25 | 7503563CB1 | 8017520CA2, |
| | | | | | 8017664CA2 |
| 6244251 | 6 | 6244251CD1 | 26 | 6244251CB1 | |
| 7503467 | 7 | 7503467CD1 | 27 | 7503467CB1 | 6262711CA2, |
| | | | | | 90050304CA2, |
| | | | | | 90050312CA2, |
| | | | | | 90050320CA2, |
| | | | | | 90050328CA2, |
| | | | | | 90050336CA2, |
| | | | | | 90050344CA2, |
| | | | | | 90050374CA2, |
| | | | | | 90050404CA2, |
| | | | | | 90050412CA2, |
| | | | | | 90050420CA2, |
| | | | | | 90050428CA2, |
| | | | | | 90050436CA2, |
| | | | | | 90050441CA2, |
| | | | | | 90050444CA2, |
| | | | | | 90050453CA2, |
| | | | | | 90050468CA2, |
| | | | | | 90050635CA2, |
| | | | | | 90066560CA2 |
| 6599034 | 8 | 6599034CD1 | 28 | 6599034CB1 | |
| 7504179 | 9 | 7504179CD1 | 29 | 7504179CB1 | |
| 71249354 | 10 | 71249354CD1 | 30 | 71249354CB1 | |
| 7505803 | 11 | 7505803CD1 | 31 | 7505803CB1 | 3524185CA2, |
| | | | | | 90179201CA2, |
| | | | | | 90179233CA2, |
| | | | | | 90179333CA2 |
| 7505804 | 12 | 7505804CD1 | 32 | 7505804CB1 | |
| 7505846 | 13 | 7505846CD1 | 33 | 7505846CB1 | 90053747CA2, |
| | | | | | 90053755CA2 |
| 55004585 | 14 | 55004585CD1 | 34 | 55004585CB1 | |
| 7506012 | 15 | 7506012CD1 | 35 | 7506012CB1 | |
| 7506212 | 16 | 7506212CD1 | 36 | 7506212CB1 | |
| 7481808 | 17 | 7481808CD1 | 37 | 7481808CB1 | |
| 7488221 | 18 | 7488221CD1 | 38 | 7488221CB1 | |
| 7505894 | 19 | 7505894CD1 | 39 | 7505894CB1 | 6262711CA2, |
| | | | | | 90050304CA2, |
| | | | | | 90050312CA2, |
| | | | | | 90050320CA2, |
| | | | | | 90050328CA2, |
| | | | | | 90050336CA2, |
| | | | | | 90050344CA2, |
| | | | | | 90050374CA2, |
| | | | | | 90050404CA2, |
| | | | | | 90050412CA2, |
| | | | | | 90050420CA2, |
| | | | | | 90050428CA2, |
| | | | | | 90050436CA2, |
| | | | | | 90050441CA2, |
| | | | | | 90050444CA2, |
| | | | | | 90050468CA2, |
| | | | | | 90050635CA2, |
| | | | | | 90066560CA2 |
| 7505901 | 20 | 7505901CD1 | 40 | 7505901CB1 | 2702495CA2 |

TABLE 2

| | | GenBank ID NO: | | |
|---|---|---|---|---|
| Polypeptide | Incyte | or PROTEOME | Probability | |
| SEQ ID NO: | Polypeptide ID | ID NO: | Score | Annotation |
| 1 | 7500521CD1 | g7271867 | 9.4E−26 | [Homo sapiens] golgi membrane protein GP73 |
| | | | | Kladney, R. D. et al. (2000) GP73, a novel Golgi-localized protein upregulated by |
| | | | | viral infection. Gene 249: 53-65. |
| 2 | 7502992CD1 | g1163174 | 2.8E−24 | [Rattus norvegicus] similar to yeast Sec6p, Swiss-Prot Accession Number |
| | | | | P32844; similar to mammalian B94, Swiss-Prot Accession Number Q03169; |
| | | | | Method: conceptual translation supplied by author. |
| | | | | Ting, A. E. et al. (1995) rSec6 and rSec8, mammalian homologs of yeast proteins |
| | | | | essential for secretion. Proc. Natl. Acad. Sci. USA 92: 9613-9617. |
| | | 426545\|SEC6 | 1.9E−25 | [Homo sapiens] [Plasma membrane] Protein with weak similarity to murine |
| | | | | Tnfip2, which is a tumor necrosis factor alpha(TNF)-induced protein. |
| | | | | Charron, A. J. et al. (2000) Compromised cytoarchitecture and polarized |
| | | | | trafficking in autosomal dominant polycystic kidney disease cells. J. Cell Biol. |
| | | | | 149: 111-124. |
| 3 | 71187173CD1 | g7920147 | 0.0 | [Homo sapiens] N-ethylmaleimide-sensitive factor |
| | | 623572\|NSF | 0.0 | [Homo sapiens] [Hydrolase; ATPase] N-ethylmaleimide-sensitive factor, an |
| | | | | ATPase involved in membrane fusion during exocytosis. |
| | | | | Hoyle, J. et al. (1996) Localization of human and mouse N-ethylmaleimide- |
| | | | | sensitive factor (NSF) gene: a two-domain member of the AAA family that is |
| | | | | involved in membrane fusion. Mamm. Genome 7: 850-852. |
| 4 | 7503143CD1 | g13752411 | 3.4E−291 | [Homo sapiens] TOB3 |
| | | 598630\|FLJ10709 | 1.8E−294 | [Homo sapiens] [Hydrolase; Protease (other than proteasomal); ATPase] |
| | | | | [Cytoplasmic; Mitochondrial] Member of the ATPases associated with various |
| | | | | cellular activities (AAA) protein family, has low similarity to SPG7 (paraplegin), |
| | | | | which is a nuclear-encoded mitochondrial metalloprotease associated with |
| | | | | hereditary spastic paraplegia (HSP). |
| 5 | 7503563CD1 | g13938372 | 7.5E−81 | [Homo sapiens] SNARE protein |
| | | 567962\|YKT6 | 6.5E−82 | [Homo sapiens] [Docking protein] [Golgi; Endoplasmic reticulum; Secretory |
| | | | | vesicles; Cytoplasmic; Plasma membrane] SNARE protein required for vesicle |
| | | | | transport between the endoplasmic reticulum and Golgi. |
| | | | | McNew, J. A. et al. (1997) Ykt6p, a prenylated SNARE essential for endoplasmic |
| | | | | reticulum-Golgi transport. J. Biol. Chem. 272: 17776-17783. |
| 6 | 6244251CD1 | g8099669 | 0.0 | [Homo sapiens] golgin-like protein |
| | | | | Gilles, F. et al. (2000) Cloning and characterization of a Golgin-related gene from |
| | | | | the large-scale polymorphism linked to the PML gene. Genomics 70: 364-374. |
| | | 599670\|GLP | 0.0 | [Homo sapiens] Protein with moderate similarity to GOLGA2 (Golgin-95), which |
| | | | | is a Golgi protein with leucine zipper and glutamate- and proline-rich tracts, and |
| | | | | an autoantigen in some autoimmune disorders. |
| | | | | Gilles, F. et al. (2000), supra. |
| 7 | 7503467CD1 | g3641674 | 7.6E−40 | [Homo sapiens] gammal-adaptin |
| | | | | Takatsu, H. et al. (1998) Identification and characterization of novel clathrin |
| | | | | adaptor-related proteins. J. Biol. Chem. 273: 24693-24700. |
| | | 334094\|AP1G1 | 6.7E−41 | [Homo sapiens] [Vesicle coat protein] [Golgi; Cytoplasmic] Gamma-adaptin 1, a |
| | | | | member of the adaptin family of proteins, promotes the formation of clathrin |
| | | | | coated vesicles and pits, involved in intracellular transport. |
| | | | | Peyrard, M. et al. (1998) Cloning, expression pattern, and chromosomal |
| | | | | assignment to 16q23 of the human ganmia-adaptin gene (ADTG). Genomics |
| | | | | 50: 275-280. |
| | | | | Takatsu, H. et al. (1998), supra. |
| 8 | 6599034CD1 | g5870426 | 3.0E−130 | [Homo sapiens] epsilon-COP protein |
| | | 428378\|COPE | 1.1E−130 | [Homo sapiens] Coatomer protein complex subunit epsilon (human leucocyte |
| | | | | vacuolar sorting protein), a putative component of the coatomer complex (COPI), |
| | | | | may be involved in vesicle transport, may have clinical significance in |
| | | | | inflammatory mediator release. |
| | | | | Guo, Q. et al. (1994) Disruptions in Golgi structure and membrane traffic in a |
| | | | | conditional lethal mammalian cell mutant are corrected by epsilon-COP. J. Cell |
| | | | | Biol. 125: 1213-1224 |
| | | | | Rajasekariah, P. et al. (1999) Molecular cloning and characterization of a cDNA |
| | | | | encoding the human leucocyte vacuolar protein sorting (h1Vps45). Int. J. |
| | | | | Biochem. Cell Biol. 31: 683-694. |
| 9 | 7504179CD1 | g202928 | 6.4E−45 | [Rattus norvegicus] clathrin-associated protein 17 |
| | | | | Kirchhausen, T. et al. (1991) AP17 and AP19, the mammalian small chains of the |
| | | | | clathrin-associated protein complexes show homology to Yap17p, their putative |
| | | | | homolog in yeast. J. Biol. Chem. 266: 11153-11157. |
| | | 340214\|AP2S1 | 6.40E−45 | [Homo sapiens] [Vesicle coat protein] [Endosome/Endosomal vesicles; Secretory |
| | | | | vesicles; Cytoplasmic; Plasma membrane] Adaptor-related protein complex 2 |
| | | | | sigma 1 subunit, associated with clathrin coated vesicles and involved in |
| | | | | intracellular transport. |
| | | | | Winterpacht, A. et al. (1996) Human CLAPS2 encoding AP17, a small chain of |
| | | | | the clathrin-associated protein complex: cDNA cloning and chromosomal |
| | | | | assignment to 19q13.2-q13.3. Cytogenet. Cell Genet. 75: 132-135 |
| | | | | Holzmann, K. et al. (1998) A novel spliced transcript of human CLAPS2 |
| | | | | encoding a protein alternative to clathrin adaptor protein AP17. Gene 220: 39-44. |
| 10 | 71249354CD1 | g2792500 | 0.0 | [Rattus norvegicus] clathrin assembly protein short form |
| | | | | Kim, H.-L. and Lee, S.-C. (1999) Exp. Mol. Med. 31: 191-196. |
| | | 298495\|PICALM | 6.4E−219 | [Homo sapiens] [Complex assembly protein] Clathrin assembly lymphoid myeloid |
| | | | | leukemia protein, binds to clathrin heavy chain (CLTC) and plays a role in coated |
| | | | | pit internalization; rearrangements in the corresponding gene are associated with |
| | | | | acute lymphoblastic and acute myeloid leukemias. |
| | | | | Vecchi, M. et al. (2001) J. Cell. Biol. 153: 1511-1517. |
| | | | | Tebar, F. et al. (1999) Mol. Biol. Cell. 10: 2687-2702. |
| | | 333520\|Rn. 10888 | 8.4E−217 | [Rattus norvegicus] [Complex assembly protein] Clathrin assembly lymphoid |
| | | | | myeloid leukemia protein, plays a role in coated pit internalization; |
| | | | | rearrangements in the corresponding human CALM gene are associated with acute |
| | | | | lymphoblastic and acute myeloid leukemias. |
| | | | | Kim, H.-L., and Kim, J. A. (2000) Exp. Mol. Med. 32: 222-226. |
| | | | | Kim, H.-L. and Lee, S.-C. (1999), supra. |
| 11 | 7505803CD1 | g2791806 | 1.8E−21 | [Mus musculus] bet3 |
| | | 323780\|Bet3 | 1.6E−22 | [Mus musculus] Protein with high similarity to S. cerevisiae BET3, which is a low |
| | | | | molecular weight subunit of Transport Protein Particle (TRAPP) complex that is |
| | | | | involved in targeting and fusion of ER to Golgi transport vesicles. |
| 12 | 7505804CD1 | g5917668 | 5.4E−70 | [Homo sapiens] cysteine-rich hydrophobic 2 CHIC2 |
| | | | | Cools, J. et al. (1999) Blood 94: 1820-1824. |
| | | 429060\|CHIC2 | 4.7E−71 | [Homo sapiens] Cysteine-rich hydrophobic protein; corresponding gene is at a |
| | | | | translocation breakpoint and undergoes fusion to ETV6 in acute myeloid |
| | | | | leukemias. |
| | | | | Cools, J. et al. (1999), supra. |
| | | | | Cools, J. et al. (2001) FEBS Lett. 492: 204-209. |
| | | 368616\|Chic1 | 3.2E−42 | [Mus musculus] Cysteine-rich hydrophobic domain 1, may playa role in brain |
| | | | | development; corresponding gene is located in the X-inactivation center and is |
| | | | | subject to X-inactivation |
| | | | | Simmler, M. C. et al. (1997) Mamm. Genome 8: 760-766. |
| 13 | 7505846CD1 | g1373146 | 4.9E−65 | [Homo sapiens] CALM |
| | | | | Dreyling, M. H. et al. (1996) Proc. Natl. Acad. Sci. USA 93: 4804-4809. |
| | | 298495\|PICALM | 4.2E−66 | [Homo sapiens] [Complex assembly protein] Clathrin assembly lymphoid myeloid |
| | | | | leukemia protein, binds to clathrin heavy chain (CLTC) and plays a role in coated |
| | | | | pit internalization; rearrangements in the corresponding gene are associated with |
| | | | | acute lymphoblastic and acute myeloid leukemias. |
| | | | | Vecchi, M. et al. (2001), supra. |
| | | | | Tebar, F. et al. (1999), supra. |
| | | 333520\|Rn. 10888 | 1.4E−65 | [Rattus norvegicus] [Complex assembly protein] Clathrin assembly lymphoid |
| | | | | myeloid leukemia protein, plays a role in coated pit internalization; |
| | | | | rearrangements in the corresponding human CALM gene are associated with acute |
| | | | | lymphoblastic and acute myeloid leukemias. |
| | | | | Kim, H.-L. and Kim, J. A. (2000), supra. |
| | | | | Kim, H.-L. and Lee, S.-C. (1999), supra. |
| 14 | 55004585CD1 | g10180266 | 0.0 | [Mus musculus] LBA |
| | | | | Wang, J. W. et al. (2001) J. Immunol. 166: 4586-4595. |
| | | 698261\|LRBA | 0.0 | [Homo sapiens] Lipopolysaccharide-responsive and beige-like anchor, a putative |
| | | | | protein-binding protein that contains WD-like repeats and a BEACH (BEige And |
| | | | | CHS) domain, may play a role in vesicle transport. |
| | | | | Wang, J. W. et al. (2001), supra. |
| | | 242600\|F10F2.1 | 0.0 | [Caenorhabditis elegans] Protein with a WD domain and a G-beta repeat; has a |
| | | | | region with high similarity to S. cerevisiae Bph1p. |
| | | | | Shea, J. E. et al. (1994) Nucleic Acids Res. 22: 5555-5564. |
| 15 | 7506012CD1 | g1929347 | 2.4E−202 | [Homo sapiens] mu-adaptin-related protein 2 |
| | | | | Wang, X. and Kilimann, M. W. (1997) FEBS Lett. 402: 57-61. |
| | | 743530\|AP4M1 | 8.1E−204 | [Homo sapiens] [Vesicle coat protein] [Golgi; Cytoplasmic; Other vesicles of the |
| | | | | secretory/endocytic pathways] Adaptor-related protein complex 4 mu 1 subunit, |
| | | | | member of the clathrin adaptor complex medium chain (mu) family, interacts with |
| | | | | tyrosine-based sorting signals and is involved in Golgi to endosome protein |
| | | | | trafficking. |
| | | | | Wang, X. and Kilimann, M. W. (1997), supra. |
| | | | | Aguilar, R. C. et al. (2001) J. Biol. Chem. 276: 13145-13152. |
| | | 580887\|Ap1m1 | 4.3E−38 | [Mus musculus] [Vesicle coat protein] [Golgi; Secretory vesicles; Cytoplasmic] |
| | | | | Medium chain 1 of the clathrin-associated protein complex Ap-1, a member of the |
| | | | | medium chain family of the clatherin adapter complex that is involved in |
| | | | | intracellular protein transport. |
| | | | | Folsch, H. et al. (2001) J. Cell Biol. 152: 595-606. |
| 16 | 7506212CD1 | g5733726 | 0.0 | [Homo sapiens] (AF169548) gamma-synergin |
| | | | | Page, L. J. et al. (1999) J. Cell Biol. 146: 993-1004. |
| | | 428468\|AP1GBP1 | 0.0 | [Homo sapiens] [Golgi; Secretory vesicles; Cytoplasmic] AP1 gamma subunit |
| | | | | binding protein 1, interacts with gamma-adaptins (AP1G1 and AP1G2) and the |
| | | | | AGEH domains of GGA proteins (GGA1, KIAA1080, KIAA0154), may be |
| | | | | involved in intracellular protein trafficking. |
| | | | | Page, L. J. et al. (1999), supra. |
| | | 712809\|Ap1gbp1 | 1.2E−219 | [Rattus norvegicus] AP1 gamma subunit binding protein 1, interacts with gamma- |
| | | | | adaptin (Ap1g1) and Scamp1, may be involved in intracellular protein trafficking. |
| | | | | Fernandez-Chacon, R. et al. (2000) Biol. Chem. 275: 12752-12756. |
| 17 | 7481808CD1 | g10801596 | 8.0E−161 | [Mus musculus] Doc2gamma |
| | | | | Fukuda, M. and Mikoshiba, K. (2000) Biochem. Biophys. |
| | | | | Res. Commun. 276: 626-632. |
| | | 624062\|Doc2g | 6.7E−162 | [Mus musculus] [Small molecule-binding protein] Double C2 protein gamma, |
| | | | | contains a Munc13-1 interacting domain (Mid) and two C2 domains, a possible |
| | | | | effector for Munc13-1 and may help regulate vesicular trafficking, highly |
| | | | | expressed in heart. |
| | | | | Fukuda, M. and Mikoshiba, K. (2000), supra. |
| | | 570448\|KIAA0985 | 4.5E−91 | [Homo sapiens] [Small molecule-binding protein] [Secretory vesicles; |
| | | | | Cytoplasmic; Plasma membrane] Rabphilin-3A, a Ca2+ and phospholipid binding |
| | | | | synaptic vesicle protein that may be involved in intracellular transport and |
| | | | | neurotransmitter release; may be a target for Rab3A small GTP binding protein. |
| | | | | Orita, S. et al. (1995) Biochem. Biophys. Res. Commun. 206: 439-448. |
| 18 | 7488221CD1 | g2827162 | 0.0 | [Rattus norvegicus] rsec15 |
| | | | | Kee, Y. et al. (1997) Proc. Natl. Acad. Sci. USA 94: 14438-14443. |
| | | 609871\|Sec15 | 0.0 | [Rattus norvegicus] Rat SEC15, a subunit of the mammalian exocyst complex, |
| | | | | may have a role in exocytosis and vesicle fusion. |
| | | | | Kee, Y. et al. (1997), supra. |
| | | 563627\|SEC15L | 0.0 | [Homo sapiens] Protein with strong similarity to rat Sec15, which is a subunit of |
| | | | | the exocyst complex that may have a role in vesicle fusion. |
| 19 | 7505894CD1 | g3641674 | 8.1E−40 | [Homo sapiens] gamma1-adaptin |
| | | | | Takatsu, H. et al. (1998), supra. |
| | | 746241\|Ap1g1 | 6.8E−41 | [Mus musculus] Gamma-adaptin 1, subunit of the Golgi adaptor, which links |
| | | | | clathrin to transmembrane proteins in coated pits and vesicles. |
| | | | | Robinson, M. S. et al. (1990) J. Cell. Biol. 111: 2319-2326. |
| | | 334094\|AP1G1 | 6.9E−41 | [Homo sapiens] [Vesicle coat protein] [Golgi; Cytoplasmic] Adaptor-related |
| | | | | protein complex 1 gamma 1 subunit, promotes the formation of clathrin coated |
| | | | | vescicles and pits for intracellular transport; deletion of the corresponding gene |
| | | | | occurs in Wilm's tumor, prostate adenocarcinomas, and hepatocelluar carcinomas. |
| | | | | Takatsu, H. et al. (1998), supra. |
| 20 | 7505901CD1 | g12803245 | 2.6E−100 | [Homo sapiens] syntaxin 4A (placental) |
| | | 341314\|STX4A | 1.2E−100 | [Homo sapiens] Syntaxin 4, broadly expressed target SNAP receptor (t-SNARE), |
| | | | | involved in targeting and exocytosis of a variety of secretory vesicles, interacts |
| | | | | with SNAP23, regulates alpha granule release in platelets. |
| | | | | Cabaniols, J. P. et al. (1999) Mol. Biol. Cell. 10: 4033-4041. |
| | | 581533\|Stx4a | 6.8E−98 | [Mus musculus] [Cytoplasmic] Syntaxin 4, broadly expressed target SNAP |
| | | | | receptor (t-SNARE), involved in targeting and exocytosis of a variety of secretory |
| | | | | vesicles via interactions with Vamp2, Snap23, Dnajc5, and other proteins, |
| | | | | regulates glucose transporter 4 (Slc2a4) trafficking. |
| | | | | Olson, A. L. et al. (1997) Mol. Cell. Biol. 17: 2425-2435. |
| | | 705044\|Stx4a | 1.4E−97 | [Rattus norvegicus] [Vesicle coat protein; Docking protein] [Basolateral plasma |
| | | | | membrane; Unspecified membrane] Syntaxin 4, broadly expressed target SNAP |
| | | | | receptor (t-SNARE), involved in targeting and exocytosis of a variety of secretory |
| | | | | vesicles via interactions with Vamp2, Rab4, and other proteins; upregulated in an |
| | | | | insulin-resistant diabetic model. |
| | | | | Maier, V. H. et al. (2000) Diabetes 49: 618-625. |

TABLE 3

| SEQ | | | Potential | Potential | | |
|---|---|---|---|---|---|---|
| ID | | Amino Acid | Phosphorylation | Glycosylation | | Analytical |
| NO: | Incyte Polypeptide ID | Residues | Sites | Sites | Signature Sequences, Domains and Motifs | Methods and Databases |
| 1 | 7500521CD1 | 380 | S36 S68 S92 S277 | N115 N150 | signal_cleavage: M1-A29 | SPSCAN |
| | | | S328 T76 T195 T312 | | | |
| | | | | | Signal Peptide: M1-A29 | HMMER |
| | | | | | Cytosolic domain: M1-R12 | TMHMMER |
| | | | | | Transmembrane domain: L13-I35 | |
| | | | | | Non-cytosolic domain: S36-L380 | |
| 2 | 7502992CD1 | 326 | S75 S110 S165 | | signal_cleavage: M1-A55 | SPSCAN |
| | | | S195 Y137 | | | |
| | | | | | PROTEIN B94 TUMOR NECROSIS FACTOR | BLAST_PRODOM |
| | | | | | INDUCED PRIMARY RESPONSE | |
| | | | | | PD025051: L92-G243 | |
| | | | | | Leucine zipper pattern: L176-L197, L183-L204 | MOTIFS |
| 3 | 71187173CD1 | 744 | S139 S356 S437 | N190 N435 | ATPase family associated with various cellular activities | HMMER_PFAM |
| | | | S460 S531 S547 | | (AAA): G255-N454, S538-S717 | |
| | | | S647 S739 T48 T114 T166 T373 | | | |
| | | | T411 T461 T476 T494 T579 | | | |
| | | | T646 Y112 Y593 | | | |
| | | | | | Cell division protein 48 (CDC48), domain 2: E98-F185 | HMMER_PFAM |
| | | | | | Cell division protein 48 (CDC48), N-terminal: A2-D86 | HMMER_PFAM |
| | | | | | AAA-protein family proteins | BLIMPS_BLOCKS |
| | | | | | BL00674: Q353-D399, N435-N454, V174-N194, V253-G274, | |
| | | | | | G296-G338 | |
| | | | | | AAA-protein family signature: D348-V424 | PROFILESCAN |
| | | | | | PROTEIN VESICULAR FUSION FUSION | BLAST_PRODOM |
| | | | | | TRANSPORT ENDOPLASMIC RETICULUM GOLGI | |
| | | | | | STACK ATP-BINDING REPEAT | |
| | | | | | PD006896: A2-K254; PD006245: L609-G737 | |
| | | | | | PROTEIN ATP-BINDING PROTEASE SUBUNIT | BLAST_PRODOM |
| | | | | | HOMOLOG REPEAT CELL DIVISION ATP- | |
| | | | | | DEPENDENT NUCLEAR | |
| | | | | | PD000092: V285-M453 | |
| | | | | | PROTEIN FUSION VESICULAR FUSION N- | BLAST_PRODOM |
| | | | | | ETHYLMALEIMIDE-SENSITIVE TRANSPORT | |
| | | | | | ENDOPLASMIC RETICULUM GOLGI STACK ATP-BINDING | |
| | | | | | PD006817: V539-L608 | |
| | | | | | AAA-PROTEIN FAMILY | BLAST_DOMO |
| | | | | | DM02248\|P46459\|2-210: A2-P211 | |
| | | | | | DM02248\|P18708\|2-210: A2-P211 | |
| | | | | | DM02248\|P46460\|2-210: A2-P211 | |
| | | | | | AAA-PROTEIN FAMILY | BLAST_DOMO |
| | | | | | DM00024\|P18708\|212-383: D212-L384 | |
| | | | | | AAA-protein family signature: I367-R385 | MOTIFS |
| 4 | 7503143CD1 | 648 | S48 S225 S263 | N266 N341 N400 | ATPase family associated with various cellullar activities | HMMER_PFAM |
| | | | S304 S369 S402 | | (AAA): N347-A543 | |
| | | | S444 S465 S516 S526 S629 T53 | | | |
| | | | T118 T173 T221 T242 T288 T292 | | | |
| | | | T338 T356 T377 T401 | | | |
| | | | T422 T612 Y70 | | | |
| | | | | | AAA-protein family proteins | BLIMPS_BLOCKS |
| | | | | | BL00674: Y345-A366, G378-R420, L433-E479, G524-A543 | |
| | | | | | PROTEIN ATPase-LIKE F54B3.3 | BLAST_PRODOM |
| | | | | | PD034475: R219-N347 TROPOMYOSIN | BLAST_DOMO |
| | | | | | DM00077\|P37709\|1104-1277: E56-K214 | |
| | | | | | TRICHOHYALIN | BLAST_DOMO |
| | | | | | DM03839\|P37709\|632-1103: E66-R227 | |
| | | | | | AAA-PROTEIN FAMILY | BLAST_DOMO |
| | | | | | DM00024\|P25694\|208-367: R346-I463 | |
| | | | | | ATP/GTP-binding site motif A (P-loop): G352-T359 | MOTIFS |
| | | | | | Growth factor and cytokines receptors family signature 1: | MOTIFS |
| | | | | | C606-W618 | |
| 5 | 7503563CD1 | 164 | S35 S51 S58 T92 | N72 | Synaptobrevin: G54-Q143 | HMMER_PFAM |
| | | | | | PROTEIN SNARE YKT6 PRENYLATED | BLAST_PRODOM |
| | | | | | TRANSMEMBRANE YKT6P ISOPRENYLATED V-SNARE B0361.8 | |
| | | | | | CHROMOSOME PD010770: M1-K53 | |
| 6 | 6244251CD1 | 702 | S53 S94 S191 S221 | N463 | Myosin tail: Q468-K493 | HMMER_PFAM |
| | | | S274 S295 S320 S455 S491 | | | |
| | | | S517 S541 S682 T12 T51 | | | |
| | | | T112 T144 T204 T256 T373 T579 | | | |
| | | | | | GOLGI STACK COILED COIL GOLGIN 95 CIS- | BLAST_PRODOM |
| | | | | | GOLGI MATRIX PROTEIN GM130 SIMILAR | |
| | | | | | PD033411: F542-D701 | |
| | | | | | PROTEIN COILED-COIL CHAIN MYOSIN REPEAT | BLAST_PRODOM |
| | | | | | HEAVY ATP-BINDING FILAMENT HEPTAD | |
| | | | | | PD000002: R240-L480, E298-E484, Q217-K464, V196-L445, | |
| | | | | | L330-E560, Q167-L409, R149-R396 | |
| | | | | | GOLGIN 95 GOLGI STACK COILED-COIL | BLAST_RODOM |
| | | | | | PD173178: E220-M282 | |
| | | | | | CIS-GOLGI MATRIX PROTEIN GM130 GOLGI | BLAST_PRODOM |
| | | | | | STACK COILED-COIL | |
| | | | | | PD180737: H626-D701 | |
| | | | | | TRICHOHYALIN | BLAST_DOMO |
| | | | | | DM03839\|P37709\|632-1103: I84-E484, Q68-E478, E103-E484, Q68-R479, R70-E478 | |
| | | | | | DM03839\|P22793\|921-1475: T144-K609, E90-R479, Q87-E484, | |
| | | | | | R70-E537, Q87-R479, Q68-R436, L210-R479 | |
| | | | | | DM03839\|\|Q07283\|91-443: Q182-E484, Q182-L449, P66-E400 | |
| | | | | | TROPOMYOSIN | BLAST_DOMO |
| | | | | | DM00077\|P37709\|1104-1277: E298-Q448, L330-E484, | |
| | | | | | E343-E484, R242-Q433 | |
| | | | | | Leucine zipper pattern: L360-L381, L367-L388, L374-L395, | MOTIFS |
| | | | | | L381-L402, L388-L409, L395-L416, L402-L416, | |
| | | | | | L409-L423, L416-L430 | |
| 7 | 7503467CD1 | 137 | S37 S76 T13 T16 | | Adaptin N terminal region: E23-V85 | HMMER_PFAM |
| | | | T19 T44 T80 Y45 | | | |
| | | | | | PROTEIN COATED SUBUNIT PITS COMPLEX | BLAST_PRODOM |
| | | | | | ADAPTIN ALPHA-ADAPTIN CLATHRIN ASSEMBLY LARGE PD001921: L7-I84 | |
| | | | | | ADAPTIN; GAMMA; YPR029C; ALPHA | BLAST_DOMO |
| | | | | | DM03711\|P22892\|2-804: A3-A126, I84-A121 | |
| | | | | | DM03711\|S49876\|5-840: L7-I84, T92-G119 | |
| | | | | | DM03711\|S54503\|1-816: L7-I84, S76-A121 | |
| 8 | 6599034CD1 | 256 | S44 S77 S95 S99 | N116 | COATOMER EPSILON SUBUNIT PROTEIN EPSILON | BLAST_PRODOM |
| | | | T218 | | COAT EPSILON-COP TRANSPORT GOLGI STACK MEMBRANE | |
| | | | | | PD017726: D17-T193, S190-A256 | |
| 9 | 7504179CD1 | 92 | T62 | | Clathrin adaptor complex small chain: M1-E92 | HMMER_PFAM |
| | | | | | Insulin family signature: D25-K80 | PROFILESCAN |
| | | | | | PROTEIN CLATHRIN ASSEMBLY SUBUNIT COAT | BLAST_PRODOM |
| | | | | | SMALL CHAIN ADAPTOR COATED PITS PD003841: F2-E92 | |
| | | | | | CLATHRIN ADAPTOR COMPLEXES SMALL CHAIN | BLAST_DOMO |
| | | | | | DM02291\|P53680\|1-141: F2-E92 | |
| | | | | | DM02291\|Q00381\|1-146: K6-E92 | |
| | | | | | DM02291\|P35181\|1-145: F2-E92 | |
| | | | | | DM02291\|Q09905\|1-145: K6-L82 | |
| | | | | | Clathrin adaptor complexes small chain signature: | MOTIFS |
| | | | | | I7-F17 | |
| 10 | 71249354CD1 | 610 | S5 S62 S128 S137 | N69 N105 N384 | ENTH (Epsin N-terminal homology) domain: G19-V141 | HMMER_PFAM |
| | | | S273 T7 T30 T161 | N445 N505 N513 | | |
| | | | T317 T386 T507 T508 T523 | | | |
| | | | | | PROTEIN CLATHRIN ASSEMBLY COAT AP180 | BLAST_PRODOM |
| | | | | | ASSOCIATED COATED PITS ALTERNATIVE | |
| | | | | | SPLICING PD014599: G420-W528, A358-H402, L354-G419 PD009526: Q4-R139 | |
| | | | | | PROTEIN ASSEMBLY CLATHRIN COAT AP180 | BLAST_PRODOM |
| | | | | | ASSOCIATED COATED PITS ALTERNATIVE SPLICING PD005811: M156-K291 | |
| | | | | | PROTEIN CLATHRIN ASSEMBLY FORM CALM | BLAST_PRODOM |
| | | | | | SHORT LONG TYPE I PD152556: Q529-M610 Cell attachment | |
| | | | | | sequence: R261-D263 | MOTIFS |
| 11 | 7505803CD1 | 53 | S11 T27 | | BET3 PROTEIN CCP1 MET1 INTERGENIC REGION | BLAST_PRODOM |
| | | | | | ZK1098.5 CHROMOSOME III | |
| | | | | | PD016734: K13-E53, M1-M14 | |
| 12 | 7505804CD1 | 137 | S44 S109 T56 T83 | | Fungal Zn(2)-Cys(6) binuclear cluster domain proteins | BLIMPS_BLOCKS |
| | | | | | BL00463: C103-E114 | |
| 13 | 7505846CD1 | 130 | S5 S62 T7 T30 | N69 | ENTH domain: G19-M130 | HMMER_PFAM |
| | | | | | PROTEIN CLATHRIN ASSEMBLY COAT AP180 | BLAST_PRODOM |
| | | | | | ASSOCIATED COATED PITS ALTERNATIVE | |
| | | | | | SPLICING | |
| | | | | | PD009526: Q4-R114 | |
| | | | | | PROTEIN CLATHRIN ASSEMBLY FORM CALM | BLAST_PRODOM |
| | | | | | SHORT LONG TYPE I | |
| | | | | | PD152556: T81-M130 | |
| 14 | 55004585CD1 | 2852 | S34 S61 S121 S278 | N163 N335 N851 | Signal Peptide: M46-V60 | HMMER |
| | | | S326 S435 S508 | N966 N992 N1013 | | |
| | | | S535 S653 S730 | N1040 N1217 | | |
| | | | S810 S993 S999 | N1310 N1346 | | |
| | | | S1005 S1015 S1084 | N1572 N1741 | | |
| | | | S1086 S1100 S1118 | N1793 N2199 | | |
| | | | S1231 S1299 S1337 | | | |
| | | | S1412 S1488 | | | |
| | | | S1562 | | | |
| | | | S1568 S1574 S1590 | | Beige/BEACH domain: T2201-R2478 | HMMER_PFAM |
| | | | S1599 S1605 S1617 | | | |
| | | | S1753 S1795 S1849 | | | |
| | | | S2028 S2039 S2053 S2054 | | | |
| | | | S2132 S2187 S2190 S2210 | | | |
| | | | S2225 S2279 S2339 S2378 | | | |
| | | | S2446 S2608 T14 T26 | | | |
| | | | T165 T259 T389 T607 T620 | | | |
| | | | T637 T686 T1014 T1033 T1068 | | | |
| | | | T1074 T1163 T1167 T1216 T1251 | | | |
| | | | T1253 T1340 | | WD domain, G-beta repeat: P2678-S2714, L2579-S2613, | HMMER_PFAM |
| | | | T1426 T1466 | | L2761-R2795, L2619-Y2659, Q2802-Y2838 | |
| | | | T1566 T1654 T1797 T1887 | | | |
| | | | T1962 T2003 T2008 T2011 | | | |
| | | | T2017 T2089 T2140 T2161 | | | |
| | | | T2183 T2201 T2240 T2388 | | | |
| | | | T2490 T2616 T2683 T2785 | | | |
| | | | Y319 Y891 Y2232 | | | |
| | | | | | PROTEIN TRANSPORT FAN FACTOR ASSOCIATED | BLAST_PRODOM |
| | | | | | WITH NSMASE ACTIVATION REPEAT WD | |
| | | | | | PD007848: G2175-R2478, E2275-F2534, Y2094-R2192, V2630-L2655 | |
| | | | | | CDC4-LIKE PROTEIN | BLAST_PRODOM |
| | | | | | PD148854: L764-R910, P252-G383, Q360-S710, K885-S1086, | |
| | | | | | V2405-G2569, S1859-L1877 | |
| | | | | | Cell attachment sequence: R1467-D1469 | MOTIFS |
| | | | | | Prokaryotic membrane lipoprotein lipid attachment site: | MOTIFS |
| | | | | | L263-C273 | |
| 15 | 7506012CD1 | 385 | S9 S10 S77 S108 | N134 | Adaptor complexes medium subunit family: F5-I385 | HMMER_PFAM |
| | | | S109 S114 S154 S198 S240 | | | |
| | | | S280 S376 T38 T57 Y34 | | | |
| | | | | | Clathrin adaptor complexes medium chain proteins | BLIMPS_BLOCKS |
| | | | | | BL00990: G25-L62, P105-N134, E184-Q217, A366-R384 | |
| | | | | | Clathrin coat assembly protein signature | BLIMPS_PRINTS |
| | | | | | PR00314: G12-L32, L107-G135, V182-G209, L254-P269 | |
| | | | | | tRNA synthetases class I | BLIMPS_PFAM |
| | | | | | PF00587: G144-F151 | |
| | | | | | PROTEIN MEDIUM CHAIN COATED PITS | BLAST_PRODOM |
| | | | | | CLATHRIN COAT ASSEMBLY SUBUNIT COMPLEX | |
| | | | | | PD002289: I16-I385, F6-M49 | |
| | | | | | CLATHRIN ADAPTOR COMPLEXES MEDIUM | BLAST_DOMO |
| | | | | | CHAIN | |
| | | | | | DM01702\|P54672\|1-438: V48-I385, M1-V47 | |
| | | | | | DM01720\|P35602\|2-421: P98-I385, V48-P75, S3-D23 | |
| | | | | | DM01702\|Q09718\|1-445: V48-I385, M1-F33 | |
| | | | | | DM01702\|P35603\|1-440: V48-R384, M1-D23 | |
| | | | | | Cell attachment sequence: R21-D23 | MOTIFS |
| 16 | 7506212CD1 | 1269 | S155 S164 S199 | N67 N221 N263 | | |
| | | | S213 S234 S235 | N268 N295 N343 | | |
| | | | S270 S469 S473 | N624 N699 N737 | | |
| | | | S483 S557 S580 | N893 N1101 | | |
| | | | S627 S665 S742 | | | |
| | | | S769 S772 S781 S789 | | | |
| | | | S792 S809 S854 | | | |
| | | | S921 S984 S990 S1005 S1024 | | | |
| | | | S1030 S1150 S1213 S1230 S1234 | | | |
| | | | T257 T314 T318 T351 | | | |
| | | | T365 T639 T731 T743 | | | |
| | | | T803 T886 T944 T971 | | | |
| | | | T1055 T1136 T1177 Y356 Y940 | | | |
| 17 | 7481808CD1 | 394 | S15 S194 S236 | | C2 domain: L267-Q355, L101-V181 | HMMER_PFAM |
| | | | S338 T141 T160 T168 T346 | | | |
| | | | | | SMRT_C2 protein kinase C conserved region 2 domain: | HMMER_SMRT |
| | | | | | A100-D214, G266-E380 | |
| | | | | | Inositol monophosphatase family proteins | BLIMPS_BLOCKS |
| | | | | | BL00629: L114-Y122 | |
| | | | | | C2 domain signature and profile: V254-V308 | PROFILESCAN |
| | | | | | C2 domain signature and profile: L88-V142 | PROFILESCAN |
| | | | | | C2 domain signature | BLIMPS_PRINTS |
| | | | | | PR00360: K311-S324, L335-D343, A282-L294 | |
| | | | | | Synaptotagmin signature | BLIMPS_PRINTS |
| | | | | | PR00399: V254-V269, V269-A282, P326-D341 | |
| | | | | | C2-DOMAIN | BLAST_DOMO |
| | | | | | DM00150\|Q06846\|558-683: E249-L372 | |
| | | | | | DM00150\|JC2473\|249-373: E249-L372 | |
| | | | | | DM00150\|Q06846\|402-526: L85-E218, G252-G352 | |
| | | | | | DM00150\|P41885\|873-1006: E249-L372 | |
| | | | | | C2 domain signature: C274-F289 | MOTIFS |
| 18 | 7488221CD1 | 804 | S35 S150 S196 | N124 N256 N425 | PROTEIN OF THE RSEC15 SUBUNIT FINAL STEP | BLAST_PRODOM |
| | | | S206 S211 S263 | N553 N570 N573 | SECRETORY PATHWAY T14P8.16 | |
| | | | S499 S509 S623 | | PD044053: L18-L721, I601-R794 | |
| | | | S766 T10 T32 T85 | | | |
| | | | T97 T127 T276 | | | |
| | | | T384 T511 T521 | | | |
| | | | T555 T581 T589 | | | |
| | | | T621 T757 T765 | | | |
| | | | Y445 Y602 | | | |
| 19 | 7505894CD1 | 137 | S37 S76 T13 T16 | | Adaptin N terminal region: E23-V85 | HMMER_PFAM |
| | | | T19 T44 T80 Y45 | | | |
| | | | | | PROTEIN COATED SUBUNIT PITS COMPLEX | BLAST_PRODOM |
| | | | | | ADAPTIN ALPHA-ADAPTIN CLATHRIN ASSEMBLY | |
| | | | | | LARGE | |
| | | | | | PD001921: L7-I84 | |
| | | | | | ADAPTIN; GAMMA; YPR029C; ALPHA | BLAST_DOMO |
| | | | | | DM03711\|P22892\|2-804: A3-A126 | |
| | | | | | DM03711\|S49876\|5-840: L7-I84, T92-G119 | |
| | | | | | DM03711\|S54503\|1-816: L7-I84, S76-A121 | |
| 20 | 7505901CD1 | 262 | S14 S15 S36 S134 | | Syntaxin: M1-A253 | HMMER_PFAM |
| | | | S169 S181 S212 | | | |
| | | | T31 T47 T194 | | | |
| | | | T226 Y80 | | | |
| | | | | | SMRT_t_SNARE: Helical region found in SNARES: | HMMER_SMRT |
| | | | | | V160-A227 | |
| | | | | | SMRT_SynN: R33-V118 | HMMER_SMRT |
| | | | | | Cytosolic domain: M1-K238 | TMHMMER |
| | | | | | Transmembrane domain: V239-V261 | |
| | | | | | Non-cytosolic domain: G262-G262 | |
| | | | | | Syntaxin/epimorphin family proteins | BLIMPS_BLOCKS |
| | | | | | BL00914: R171-G220 | |
| | | | | | SYNTAXIN COILED-COIL TRANSMEMBRANE | BLAST_PRODOM |
| | | | | | TRANSPORT NEUROTRANSMITTER PROTEIN 1A | |
| | | | | | NEURON-SPECIFIC ANTIGEN | |
| | | | | | PD001014: D3-I256 | |
| | | | | | EPIMORPHIN FAMILY | BLAST_DOMO |
| | | | | | DM01996\|S52726\|32-297: E17-G262 | |
| | | | | | DM01996\|JU0136\|23-288: D3-V260 | |
| | | | | | DM01996\|P32856\|23-287: S36-V260 | |
| | | | | | DM01996\|D48213\|26-289: I48-V260 | |
| | | | | | Syntaxin/epimorphin family signature: R171-I210 | MOTIFS |

TABLE 4

| Polynucleotide | |
|---|---|
| SEQ ID NO:/ | |
| Incyte ID/ | |
| Sequence Length | Sequence Fragments |
| 21/7500521CB1/2251 | 1-496, 13-242, 13-323, 13-562, 13-577, 21-613, 26-625, 29-477, 29-779, 30-612, 35-272, 42-538, 69-758, 80-714, |
| | 98-592, 100-748, 100-757, 100-787, 100-789, 100-888, 100-894, 100-895, 100-918, 100-921, 100-930, 100-974, 103-921, |
| | 170-608, 177-736, 182-805, 199-862, 233-772, 233-863, 268-891, 280-2251, 287-922, 319-862, 323-502, 323-764, |
| | 338-817, 340-547, 340-584, 340-806, 340-828, 340-910, 344-563, 356-1001, 380-1055, 380-1056, 380-1111, |
| | 380-1135, 380-1150, 380-1172, 380-1195, 381-883, 381-1059, 381-1124, 381-1141, 381-1232, 382-1065, 383-1151, |
| | 402-1062, 451-1047, 481-717, 513-804, 537-1238, 591-1239, 596-1226, 604-875, 604-932, 615-875, 622-865, 653-1142, |
| | 694-1267, 700-996, 720-1024, 732-874, 806-1065, 809-1017, 809-1024, 809-1029, 809-1378, 858-1121, 959-1693, |
| | 977-1256, 977-1604, 1003-1287, 1006-1815, 1008-1815, 1011-1818, 1042-1864, 1084-1654, 1137-1391, 1137-1684, |
| | 1142-1387, 1157-1721, 1199-1526, 1331-2017, 1376-1543, 1380-1842, 1380-1945, 1382-1736, 1397-1629, |
| | 1397-1647, 1414-1710, 1520-1786, 1549-2251, 1550-1829, 1555-1872, 1609-1851, 1647-1968, 1701-1862, 1718-1989, |
| | 1729-1851, 1738-1851, 1738-1996, 1762-1846, 1781-2036, 1781-2067 |
| 22/7502992CB1/ | 1-587, 1-594, 293-673, 437-962, 524-793, 596-1055, 881-1567, 998-1230, 1024-1314, 1115-1457, 1190-1434, 1190-1775, |
| 1775 | 1191-1333, 1191-1432, 1191-1564, 1191-1586, 1191-1619, 1191-1703, 1191-1711, 1191-1722, 1191-1734, |
| | 1191-1751, 1191-1770, 1191-1773, 1191-1774, 1191-1775, 1194-1775, 1195-1775, 1202-1487, 1215-1763, 1292-1775, |
| | 1297-1468, 1298-1775, 1337-1584, 1337-1775, 1340-1629, 1341-1602, 1345-1485, 1370-1774 |
| 23/71187173CB1/ | 1-452, 1-653, 2-564, 6-522, 9-437, 9-774, 10-278, 10-536, 17-588, 17-693, 18-534, 19-271, 20-615, 23-242, 23-516, |
| 3959 | 23-658, 24-244, 25-175, 28-625, 29-658, 31-819, 40-241, 40-406, 46-311, 46-334, 46-347, 47-928, 48-398, 51-597, |
| | 51-629, 55-441, 61-142, 117-760, 385-679, 385-834, 385-933, 411-1127, 420-1011, 469-709, 472-976, 485-736, 553-827, |
| | 580-1207, 632-843, 675-1266, 692-1331, 696-1319, 707-1319, 745-1024, 763-1384, 767-1247, 777-1331, 777-1502, |
| | 809-1046, 814-1038, 814-1422, 850-1579, 854-1541, 870-1407, 894-1500, 897-1475, 912-1527, 940-1192, |
| | 997-1674, 1005-1623, 1009-1279, 1027-1291, 1028-1652, 1031-1596, 1037-1691, 1056-1442, 1063-1324, 1068-1767, |
| | 1092-1742, 1094-1726, 1096-1785, 1110-1425, 1118-1542, 1132-1447, 1139-1760, 1177-1437, 1179-1705, |
| | 1185-1744, 1196-1887, 1197-1757, 1198-1473, 1227-1789, 1278-1939, 1280-1918, 1283-1942, 1299-2040, 1304-1909, |
| | 1304-1983, 1360-1914, 1363-1776, 1409-2013, 1417-1654, 1421-1964, 1443-2090, 1444-1703, 1444-1710, |
| | 1444-1954, 1445-1984, 1451-2055, 1456-2075, 1485-2045, 1491-2146, 1504-1878, 1528-2179, 1534-2254, 1546-2074, |
| | 1555-2177, 1557-2225, 1565-1746, 1568-2165, 1579-2124, 1583-1983, 1587-2165, 1603-2326, |
| | 1605-2243, 1621-1893, 1639-2231, 1641-1928, 1703-1940, 1706-1962, 1715-1975, 1719-2318, 1721-2001, 1721-2153, |
| | 1721-2381, 1737-2222, 1761-2416, 1762-1955, 1762-1964, 1768-2417, 1775-2037, 1781-2277, 1794-2042, |
| | 1816-2333, 1832-2378, 1841-2015, 1844-2109, 1850-2045, 1868-2394, 1872-2440, 1912-2022, 1922-2142, 1926-2173, |
| | 1941-2177, 1944-2220, 1950-2476, 1966-2439, 1976-2623, 1996-2464, 2005-2355, 2006-2531, 2068-2446, |
| | 2084-2454, 2093-2609, 2104-2397, 2118-2356, 2118-2358, 2118-2360, 2119-2735, 2152-2704, 2163-2256, 2169-2789, |
| | 2179-2572, 2190-2664, 2194-2458, 2202-2441, 2202-2442, 2202-2817, 2225-2468, 2233-2758, 2234-2758, |
| | 2257-2564, 2263-2799, 2267-2718, 2274-2838, 2290-2539, 2301-2640, 2301-2847, 2310-3028, 2310-3049, 2317-2599, |
| | 2317-2881, 2318-2574, 2319-2570, 2324-2768, 2349-2809, 2366-3126, 2380-2624, 2386-2921, 2389-2627, |
| | 2391-2651, 2421-2775, 2454-2737, 2455-3119, 2476-3022, 2481-3047, 2498-2778, 2503-3042, 2504-2794, 2504-2817, |
| | 2529-2764, 2540-2920, 2573-2876, 2580-2828, 2580-3161, 2581-2826, 2586-3247, 2605-3258, 2606-3098, |
| | 2679-2928, 2700-2993, 2705-3347, 2713-3340, 2718-3260, 2719-3453, 2768-3338, 2772-3047, 2772-3060, |
| | 2784-3267, 2795-3313, 2796-3353, 2805-3453, 2812-3126, 2826-3362, 2839-3374, 2881-3334, 2886-3448, 2889-3336, |
| | 2889-3338, 2889-3370, 2898-3483, 2901-3165, 2904-3529, 2920-3497, 2931-3171, 2948-3390, 2950-3470, |
| | 2954-3478, 2962-3609, 2967-3209, 2967-3321, 2967-3547, 2968-3215, 2977-3271, 2991-3238, 2991-3252, 2998-3531, |
| | 3002-3391, 3028-3280, 3093-3757, 3099-3504, 3112-3843, 3121-3890, 3133-3874, 3134-3418, 3153-3369, |
| | 3175-3603, 3194-3709, 3206-3791, 3211-3866, 3215-3643, 3215-3646, 3215-3666, 3215-3680, 3218-3459, 3218-3709, |
| | 3223-3427, 3223-3726, 3225-3750, 3227-3837, 3229-3886, 3230-3890, 3233-3924, 3243-3592, 3247-3908, |
| | 3252-3877, 3267-3895, 3271-3534, 3283-3543, 3293-3904, 3303-3890, 3330-3597, 3340-3845, 3363-3959, 3371-3881, |
| | 3382-3938, 3403-3611, 3407-3670, 3410-3945, 3422-3948, 3441-3915, 3442-3915, 3448-3915, 3451-3915, |
| | 3452-3917, 3454-3674, 3454-3913, 3455-3915, 3456-3914, 3456-3915, 3456-3916, 3457-3918, 3458-3922, 3459-3917, |
| | 3460-3661, 3461-3915, 3463-3917, 3463-3938, 3464-3915, 3464-3916, 3465-3914, 3467-3914, 3469-3919, |
| | 3472-3917, 3477-3920, 3477-3929, 3489-3917, 3492-3723, 3495-3921, 3510-3935, 3511-3915, 3512-3717, |
| | 3516-3912, 3519-3915, 3526-3915, 3541-3917, 3561-3783, 3566-3908, 3577-3915, 3590-3808, 3600-3837, 3666-3894, |
| | 3666-3909, 3666-3919, 3713-3915, 3756-3948 |
| 24/7503143CB1/ | 1-680, 11-720, 22-752, 27-798, 27-824, 31-738, 32-753, 32-908, 33-631, 35-665, 36-732, 37-536, 37-625, 37-695, |
| 2460 | 37-773, 37-778, 37-804, 41-269, 41-736, 41-749, 45-586, 45-656, 55-836, 59-98, 76-808, 113-625, 123-579, 123-660, |
| | 123-676, 123-682, 123-685, 123-755, 123-774, 123-801, 123-825, 182-723, 182-986, 184-535, 189-525, 196-658, |
| | 206-869, 231-1132, 238-959, 247-993, 247-1072, 274-828, 307-865, 321-589, 326-973, 344-1076, 356-1150, |
| | 360-823, 372-687, 372-931, 372-1014, 372-1048, 382-1152, 418-634, 425-1119, 426-1115, 540-818, 540-981, 704-976, |
| | 823-1049, 823-1442, 856-1142, 856-1389, 860-1105, 930-1138, 1009-1254, 1009-1619, 1014-1259, 1108-1249, |
| | 1216-1464, 1465-1653, 1552-1851, 1558-1767, 1558-2146, 1602-1849, 1604-1840, 1891-2460, 1894-2138, |
| | 1943-2450, 2025-2289, 2025-2447, 2120-2391, 2123-2440, 2149-2430 |
| 25/7503563CB1/ | 1-346, 1-478, 1-493, 1-516, 1-518, 1-606, 1-668, 1-745, 8-244, 8-432, 10-309, 20-467, 23-408, 23-489, 25-480, 25-745 |
| 745 | 26-436, 26-449, 26-488, 26-499, 27-370, 27-668, 38-366, 59-491, 60-250, 74-389, 74-465, 74-489, 74-518, 101-387, |
| | 101-402, 121-482, 163-347, 163-424, 170-424, 190-460, 242-668, 260-421, 278-519, 369-515, 378-516, 518-709, |
| | 518-728, 520-742, 528-745, 555-745, 563-745, 576-745, 599-745, 602-716, 673-745 |
| 26/6244251CB1/ | 1-441, 358-2499, 876-1149, 876-1153, 876-1269, 925-1262, 930-1552, 931-1145, 933-1267, 1040-1269, 1051-1269, |
| 2738 | 1248-1451, 1248-1796, 1577-1667, 1881-2525, 2232-2703, 2296-2730, 2305-2730, 2417-2738 |
| 27/7503467CB1/ | 1-110, 1-215, 1-223, 1-621, 1-623, 1-2503, 14-717, 19-370, 19-462, 19-543, 19-591, 19-690, 20-688, 20-782, 63-844, |
| 2509 | 94-774, 111-239, 112-239, 280-718, 280-719, 280-722, 280-761, 284-930, 287-722, 301-551, 313-514, 351-894, |
| | 356-598, 358-633, 369-615, 369-658, 389-994, 399-1069, 407-723, 414-723, 417-609, 430-622, 461-798, 473-580, |
| | 481-1317, 494-747, 505-776, 510-1136, 511-775, 554-1095, 554-1172, 571-1058, 587-856, 594-841, 605-874, |
| | 610-1196, 617-948, 667-1466, 669-1461, 672-826, 697-1310, 697-1436, 712-968, 712-973, 712-1123, 724-1232, |
| | 761-1198, 763-1144, 763-1208, 766-1531, 772-1196, 775-1053, 776-1025, 792-1224, 792-1232, 801-1578, 824-1056, |
| | 846-1125, 865-1423, 868-1665, 879-1126, 901-1566, 905-1183, 905-1210, 905-1499, 905-1553, 905-1571, |
| | 906-1114, 906-1195, 910-1457, 912-1578, 954-1522, 955-1608, 961-1208, 967-1210, 989-1556, 994-1547, 1014-1252, |
| | 1015-1287, 1017-1608, 1017-1611, 1033-1583, 1073-1581, 1097-1480, 1140-1624, 1142-1624, 1144-1624, |
| | 1151-1357, 1151-1647, 1159-1618, 1163-1622, 1179-1611, 1181-1624, 1187-1624, 1188-1897, 1189-1622, 1193-1621, |
| | 1199-1620, 1205-1624, 1210-1624, 1210-1626, 1212-1621, 1213-1617, 1213-1621, 1234-1620, |
| | 1245-1487, 1246-1617, 1248-1622, 1253-1617, 1254-1533, 1259-1615, 1260-1621, 1274-1869, 1285-1621, 1286-1484, |
| | 1312-1618, 1330-1602, 1330-1621, 1335-1621, 1343-1621, 1356-1621, 1360-1622, 1376-1957, 1387-1620, |
| | 1388-1608, 1388-1623, 1388-1624, 1406-1686, 1416-1511, 1425-2162, 1438-1620, 1449-1622, 1450-1620, 1461-2039, |
| | 1533-1763, 1540-1621, 1552-1797, 1582-1811, 1582-2110, 1590-1758, 1619-1846, 1619-2157, 1655-2176, |
| | 1655-2299, 1716-2329, 1721-1891, 1721-2267, 1758-2219, 1825-2509, 1872-2136, 1872-2352, 1967-2488, 1989-2485, |
| | 2022-2271, 2041-2340, 2046-2487, 2047-2488, 2060-2485, 2065-2491, 2075-2485, 2076-2485, 2087-2345, |
| | 2092-2435, 2128-2490, 2179-2415, 2193-2494, 2200-2490, 2230-2475, 2230-2488, 2245-2488, 2343-2485 |
| 28/6599034CB1/ | 1-236, 12-340, 13-193, 13-295, 18-287, 75-294, 159-701, 267-514, 290-966, 431-966, 434-942, 438-914, 608-949 |
| 966 | |
| 29/7504179CB1/ | 1-69, 1-95, 1-794, 126-423, 128-379, 129-393, 131-414, 136-392, 151-444, 151-445, 152-429, 153-404, 153-407, |
| 820 | 206-452, 208-490, 223-377, 223-445, 223-449, 223-452, 223-457, 223-478, 223-491, 223-537, 223-564, 223-695, |
| | 223-724, 223-750, 223-756, 223-796, 223-799, 224-759, 226-515, 226-767, 226-793, 228-409, 228-493, 228-502, |
| | 228-507, 228-510, 228-512, 228-524, 228-732, 228-773, 237-527, 238-446, 238-708, 240-360, 240-545, 241-734, |
| | 242-524, 245-767, 256-688, 256-774, 257-683, 257-791, 258-496, 258-517, 263-742, 264-528, 269-531, 269-779, |
| | 272-820, 273-481, 273-525, 280-798, 281-699, 289-790, 289-796, 290-500, 291-567, 291-614, 291-774, 292-502, |
| | 292-541, 295-553, 301-578, 301-584, 301-593, 302-698, 302-779, 304-559, 305-779, 309-781, 310-532, 311-545, |
| | 311-804, 312-548, 313-587, 313-657, 313-810, 314-729, 314-736, 314-799, 316-779, 317-595, 317-797, 319-574, |
| | 322-808, 323-775, 325-656, 325-780, 326-670, 327-773, 328-589, 328-779, 328-799, 332-529, 335-774, 336-583, |
| | 336-779, 336-780, 337-538, 337-599, 338-779, 338-791, 339-779, 339-792, 340-777, 342-779, 342-795, 344-558, |
| | 344-602, 344-707, 345-778, 347-775, 350-782, 351-767, 351-777, 351-779, 352-779, 356-573, 357-614, |
| | 360-774, 361-779, 362-599, 366-477, 366-595, 366-627, 367-775, 367-779, 368-607, 368-619, 368-763, 368-777, |
| | 369-655, 369-804, 370-565, 371-775, 371-778, 372-597, 372-638, 372-774, 372-782, 377-624, 377-630, 377-763, |
| | 378-656, 379-774, 381-780, 384-678, 384-779, 385-680, 385-775, 386-782, 388-809, 389-534, 391-623, 393-818, |
| | 397-777, 398-777, 404-807, 405-642, 406-773, 406-774, 406-795, 407-663, 407-784, 407-806, 410-776, 413-755, |
| | 414-645, 421-666, 421-675, 421-699, 424-677, 424-693, 425-779, 427-779, 428-773, 429-779, 429-784, 433-774, |
| | 434-783, 438-779, 439-571, 439-763, 447-773, 450-698, 454-733, 454-779, 456-558, 469-774, 485-779, 486-707, |
| | 493-746, 501-779, 505-701, 505-739, 505-783, 506-712, 506-800, 507-772, 507-777, 510-779, 514-601, 514-779, |
| | 517-774, 520-786, 522-779, 529-779, 529-783, 532-715, 533-733, 548-805, 552-774, 554-777, 573-783, 578-745, |
| | 580-754, 581-820, 585-778, 596-779, 611-820, 613-793, 615-820, 620-737, 625-820, 640-779, 642-763, 644-792, |
| | 646-779, 647-820, 668-783, 682-820, 699-777, 727-798 |
| 30/71249354CB1/ | 1-241, 2-269, 2-288, 13-841, 27-788, 27-793, 27-869, 32-841, 110-699, 131-651, 267-895, 268-776, 269-918, 284-525, |
| 3709 | 284-851, 309-869, 315-968, 317-897, 322-818, 329-942, 336-963, 350-578, 354-755, 360-1211, 410-582, 424-1245, |
| | 426-881, 452-894, 597-1226, 630-1291, 633-1474, 635-1204, 640-1313, 646-1342, 656-1226, 667-893, 667-1002, |
| | 698-1274, 721-1353, 745-1383, 814-1459, 834-1283, 1055-1291, 1055-1558, 1126-1303, 1155-1660, 1155-1781, |
| | 1184-1834, 1185-1427, 1185-1768, 1207-1730, 1231-1465, 1233-1622, 1234-1702, 1246-1543, 1280-1468, |
| | 1296-1910, 1308-1816, 1317-1938, 1328-1976, 1335-1573, 1356-1894, 1385-1669, 1395-1615, 1410-1997, 1429-1973, |
| | 1466-1706, 1475-1691, 1475-2061, 1475-2071, 1479-1618, 1502-1534, 1507-1804, 1509-2027, 1539-2149, |
| | 1550-2116, 1551-2019, 1578-1705, 1600-2223, 1611-2256, 1623-1887, 1624-1902, 1630-1901, 1656-1925, 1677-2180, |
| | 1687-1884, 1720-1970, 1789-2239, 1816-2062, 1817-2099, 1820-2068, 1833-2095, 1847-2113, 1873-2137, |
| | 1874-2127, 1876-2149, 1877-2164, 1895-2301, 1896-2181, 1896-2183, 1911-2184, 1927-2204, 1933-2180, 1952-2403, |
| | 1979-2236, 1992-2252, 2032-2443, 2138-2535, 2164-2406, 2165-2401, 2184-2470, 2256-2494, |
| | 2257-2607, 2257-2811, 2307-2499, 2367-2578, 2391-2617, 2410-2672, 2439-2856, 2440-2716, 2453-2635, 2455-3283, |
| | 2464-2697, 2501-2717, 2514-2764, 2677-2910, 2680-2904, 2680-3170, 2733-2945, 2745-2967, 2751-3080, |
| | 2753-2958, 2837-3504, 2840-3505, 2875-3078, 2875-3125, 2879-3451, 2897-3505, 2916-3495, 2959-3214, 2967-3172, |
| | 2969-3209, 3032-3299, 3032-3463, 3032-3512, 3054-3512, 3085-3307, 3085-3529, 3105-3503, 3106-3381, |
| | 3149-3391, 3171-3390, 3196-3446, 3196-3447, 3196-3448, 3196-3525, 3357-3709 |
| 31/7505803CB1/ | 1-147, 9-440, 16-447, 16-461, 25-346, 145-312, 145-357, 145-371, 145-438, 145-461, 147-363, 151-397, 152-396, |
| 461 | 156-430, 165-435, 171-439, 193-439, 206-436, 206-439, 221-439, 240-439, 275-362, 286-439, 306-439 |
| 32/7505804CB1/ | 1-249, 1-261, 1-1254, 9-281, 14-280, 15-145, 21-271, 29-255, 37-262, 37-450, 39-395, 40-260, 41-288, 43-266, 47-242, |
| 1254 | 56-326, 70-346, 95-312, 135-432, 156-563, 206-353, 236-487, 300-563, 320-708, 385-826, 564-786, 564-806, |
| | 564-816, 564-822, 564-838, 577-1032, 589-1032, 593-839, 594-837, 598-839, 599-859, 679-1008, 692-950, 711-977, |
| | 733-856, 758-962, 843-1032, 872-1030, 874-971, 874-1033, 878-1004, 894-1036, 959-1212, 961-1109, 961-1208, |
| | 1010-1254 |
| 33/7505846CB1/ | 1-282, 1-1109, 15-111, 15-254, 15-290, 40-161, 40-737, 40-742, 45-273, 45-380, 48-316, 48-339, 52-335, 56-314, |
| 1176 | 57-501, 61-308, 259-750, 261-750, 297-521, 297-538, 317-559, 356-608, 416-608, 423-595, 608-822, 608-846, 608-847, |
| | 608-860, 608-879, 608-1016, 608-1118, 612-782, 614-855, 614-930, 625-880, 626-861, 626-958, 633-712, 639-892, |
| | 643-909, 643-911, 644-956, 649-961, 670-921, 671-944, 673-986, 688-946, 694-1165, 714-964, 718-976, 722-841, |
| | 725-1009, 737-978, 737-979, 737-1004, 738-974, 738-979, 743-1176, 748-1002, 757-1044, 759-993, 771-870, |
| | 783-1058, 784-953, 796-1062, 803-1099, 808-1049, 815-1066, 834-1068, 834-1089, 842-1099, 843-996, 880-1073 |
| 34/55004585CB1/ | 1-754, 236-796, 524-951, 671-882, 672-1222, 1154-1665, 1154-1675, 1154-1707, 1154-1712, 1572-2285, 2121-2461, |
| 9050 | 2121-6209, 2342-2682, 2370-2520, 2574-3219, 3883-4384, 3883-4386, 4086-4666, 4090-4359, 4107-4270, |
| | 4157-4447, 4218-4790, 4265-4425, 4694-4943, 4880-4932, 4960-5259, 5059-5184, 5159-5346, 5159-5391, 5159-5626, |
| | 5415-6002, 5594-6209, 5929-6294, 6006-6419, 6072-6700, 6073-6700, 6148-6625, 6273-6895, 6387-6929, |
| | 6501-6636, 6502-6849, 6502-6864, 6502-6928, 6502-6938, 6502-6970, 6552-6785, 6575-6772, 6581-7240, 6670-7309, |
| | 6781-7042, 6784-7257, 6784-8574, 6786-7292, 6786-7493, 6891-7603, 6892-7267, 6914-7006, 7160-7643, |
| | 7276-7940, 7310-7723, 7519-8023, 7675-7818, 7702-7970, 7732-8118, 7744-8127, 7773-8403, 7801-8305, 7818-8359, |
| | 7823-8363, 7866-8045, 7881-8163, 7904-7952, 7946-8162, 8003-8543, 8043-8531, 8054-8580, 8086-8654, |
| | 8158-8986, 8209-8583, 8267-8716, 8290-8545, 8294-8899, 8298-8855, 8315-8638, 8329-8592, 8329-8620, 8333-8591, |
| | 8372-8620, 8372-8949, 8381-8591, 8406-8861, 8407-9050, 8408-8993, 8424-8762, 8473-8543, 8491-8984, |
| | 8492-9046, 8503-8801, 8527-8801 |
| 35/7506012CB1/ | 1-282, 1-294, 5-1587, 15-265, 91-294, 93-519, 115-389, 125-382, 133-392, 133-406, 149-720, 190-448, 293-449, |
| 1605 | 293-762, 293-847, 293-922, 298-433, 303-1034, 308-934, 318-878, 323-809, 332-894, 333-611, 337-554, 339-616, |
| | 350-1006, 350-1021, 359-945, 370-1049, 437-899, 464-1028, 465-920, 473-1072, 484-772, 491-1071, 491-1131, |
| | 494-670, 495-1055, 498-1152, 508-1020, 510-968, 522-777, 539-928, 539-1038, 542-985, 544-1099, 549-1112, 566-815, |
| | 566-1033, 582-710, 589-1050, 608-870, 612-1010, 614-1216, 632-1246, 635-1083, 638-996, 689-943, 701-1316, |
| | 713-1279, 730-1294, 748-809, 772-1423, 778-1202, 794-1034, 799-1501, 823-1440, 825-1497, 826-1490, 838-1169, |
| | 842-1382, 857-1087, 868-1153, 904-1566, 905-1268, 925-1198, 928-1556, 938-1555, 945-1413, 972-1253, |
| | 984-1274, 1051-1321, 1051-1329, 1070-1605, 1077-1471, 1081-1403, 1088-1561, 1109-1526, 1110-1309, 1119-1358, |
| | 1122-1567, 1124-1561, 1126-1567, 1153-1569, 1159-1574, 1165-1292, 1170-1485, 1170-1561, 1171-1592, |
| | 1172-1595, 1182-1525, 1202-1557, 1225-1565, 1264-1603, 1269-1599, 1271-1585, 1278-1602, 1294-1605, 1314-1588, |
| | 1329-1598, 1331-1527, 1480-1563, 1493-1599 |
| 36/7506212CB1/ | 1-250, 1-4896, 9-346, 9-424, 9-460, 9-473, 22-264, 24-258, 56-249, 58-313, 60-683, 473-642, 652-1212, 680-921, |
| 5038 | 680-1330, 1019-1808, 1077-1808, 1094-1808, 1183-1848, 1192-1832, 1210-1848, 1219-1745, 1278-1847, 1354-1984, |
| | 1374-1943, 1404-1994, 1692-1943, 1770-2050, 1872-2105, 1872-2363, 1891-2514, 1926-2194, 2037-2606, |
| | 2140-2377, 2244-2400, 2256-2513, 2279-2889, 2386-2891, 2449-2853, 2589-3156, 2661-2853, 2661-2939, 2676-2952, |
| | 2688-3075, 2734-2951, 2734-3116, 2734-3223, 2734-3227, 2734-3261, 2734-3286, 2734-3373, 2800-3368, |
| | 2800-3372, 2803-3027, 2826-3319, 2826-3393, 2827-3021, 2839-3439, 2858-3140, 2896-3167, 2913-3176, 2984-3243, |
| | 2992-3890, 3051-3261, 3056-3648, 3061-3731, 3078-3529, 3085-3299, 3090-3890, 3107-3890, 3110-3683, |
| | 3117-3340, 3188-3890, 3206-3352, 3206-3442, 3232-3683, 3234-3831, 3243-3727, 3262-3750, 3316-3921, 3340-3837, |
| | 3341-3615, 3341-3624, 3348-3882, 3369-3654, 3387-3822, 3409-3924, 3422-3675, 3422-3711, 3422-3772, |
| | 3422-3933, 3422-3985, 3456-3999, 3462-4030, 3463-3923, 3488-4185, 3492-3936, 3492-4019, 3567-3727, 3577-4344, |
| | 3592-4155, 3619-3985, 3626-4247, 3638-4213, 3641-4114, 3649-4173, 3697-3984, 3710-4327, |
| | 3719-4017, 3734-4160, 3745-4324, 3755-4296, 3769-4337, 3776-4324, 3776-4330, 3783-4055, 3804-4344, 3822-4342, |
| | 3844-4336, 3849-4341, 3862-4344, 3875-4343, 3875-4344, 3879-4342, 3882-4344, 3885-4340, 3886-4344, |
| | 3891-4333, 3891-4344, 3895-4160, 3898-4341, 3900-4341, 3900-4344, 3902-4313, 3905-4266, 3905-4341, 3905-4344, |
| | 3909-4333, 3918-4320, 3925-4175, 3925-4263, 3928-4183, 3935-4341, 3948-4268, 3954-4222, 3962-4341, |
| | 3962-4342, 3992-4335, 3996-4337, 3996-4344, 3998-4270, 3998-4331, 3998-4341, 3998-4344, 4000-4341, 4008-4344, |
| | 4016-4338, 4016-4342, 4031-4239, 4047-4755, 4121-4319, 4122-4302, 4122-4344, 4148-4341, 4177-4341, |
| | 4177-4343, 4234-4896, 4292-4344, 4475-4622, 4475-4672, 4475-4705, 4475-4710, 4475-4716, 4475-4728, 4475-4731, |
| | 4475-4751, 4477-4677, 4480-4747, 4495-4713, 4498-4734, 4523-4846, 4531-4807, 4543-5018, 4543-5038, |
| | 4544-4804, 4550-4803, 4552-4827, 4574-4692, 4590-4851, 4595-4881, 4627-4890, 4634-4874, 4647-4889, 4649-4908 |
| 37/7481808CB1/ | 1-850, 3-851, 121-851, 129-851, 142-850, 189-851, 245-850, 583-1890, 754-1372, 961-1214, 1215-1372, 1429-1482, |
| 2083 | 1429-1580, 1429-1581, 1429-1585, 1429-1623, 1429-1629, 1429-1638, 1477-1636, 1485-1662, 1491-1844, |
| | 1562-1780, 1581-1662, 1581-1829, 1591-2063, 1618-2061, 1663-1791, 1663-1977, 1668-1822, 1756-2083 |
| 38/7488221CB1/ | 1-287, 7-679, 8-2638, 14-589, 20-312, 22-585, 23-237, 26-269, 49-656, 50-446, 65-191, 65-646, 134-617, 147-905, |
| 3615 | 246-377, 355-1074, 365-598, 365-633, 365-854, 367-938, 433-938, 433-984, 434-1206, 486-680, 630-1242, 653-1321, |
| | 672-888, 677-815, 817-1314, 850-1412, 882-1289, 885-1173, 902-1647, 1085-1458, 1101-1588, 1246-1960, |
| | 1256-1766, 1256-1860, 1268-1851, 1376-1819, 1390-1819, 1438-1735, 1520-2122, 1548-1692, 1549-2110, 1552-1862, |
| | 1581-1863, 1606-2158, 1657-1918, 1657-1939, 1680-2246, 1694-2246, 1701-2246, 1727-1974, 1738-2251, |
| | 1777-2302, 1795-2035, 1859-2116, 1954-2592, 1973-2101, 1973-2346, 1979-2205, 2001-2318, 2005-2124, 2016-2342, |
| | 2017-2604, 2026-2199, 2110-2303, 2113-2592, 2116-2620, 2116-2629, 2184-2638, 2186-2638, 2187-2638, |
| | 2189-2347, 2189-2490, 2189-2555, 2189-2584, 2189-2599, 2189-2621, 2189-2636, 2189-2690, 2189-2710, 2189-2772, |
| | 2192-2638, 2192-2818, 2194-2762, 2217-2801, 2268-2804, 2317-2534, 2317-2539, 2317-2997, 2347-2630, |
| | 2360-2625, 2423-2932, 2426-2586, 2434-2854, 2450-3094, 2456-2727, 2462-3027, 2542-2791, 2555-2808, 2558-3228, |
| | 2634-2828, 2634-3108, 2645-2891, 2645-3072, 2659-2874, 2661-2947, 2662-2955, 2669-2924, |
| | 2679-3286, 2685-2982, 2690-3307, 2709-3089, 2750-3510, 2753-3017, 2756-3332, 2757-3033, 2761-2944, 2762-3027, |
| | 2765-3368, 2766-3081, 2773-3020, 2775-3383, 2805-3036, 2809-3489, 2810-3416, 2851-3462, 2856-3278, |
| | 2890-3557, 2894-3503, 2937-3509, 2938-3193, 2939-3220, 2957-3525, 2961-3210, 2969-3260, 2970-3519, 2979-3524, |
| | 2991-3527, 2991-3569, 2995-3580, 2996-3254, 3019-3223, 3019-3279, 3026-3533, 3028-3565, 3029-3526, |
| | 3031-3527, 3041-3520, 3044-3284, 3057-3580, 3060-3569, 3072-3339, 3081-3314, 3087-3615, 3088-3610, 3090-3525, |
| | 3104-3568, 3107-3565, 3116-3569, 3120-3565, 3122-3569, 3136-3564, 3137-3571, 3139-3564, 3142-3563, |
| | 3155-3568, 3174-3424, 3174-3452, 3175-3442, 3176-3373, 3181-3394, 3236-3482, 3287-3577, 3325-3534, 3330-3495, |
| | 3356-3569, 3370-3592, 3390-3564, 3398-3568, 3447-3569, 3456-3559 |
| 39/7505894CB1/ | 1-110, 1-215, 1-223, 1-621, 1-623, 1-1194, 14-717, 19-370, 19-462, 19-514, 19-543, 19-636, 20-688, 20-782, 63-844, |
| 1194 | 94-738, 111-239, 112-239, 280-718, 280-719, 280-722, 280-761, 284-829, 287-722, 301-551, 313-514, 351-894, |
| | 356-598, 358-633, 369-615, 369-658, 389-996, 407-723, 414-723, 417-609, 430-622, 461-798, 473-580, 494-747, |
| | 496-747, 505-776, 510-1114, 554-1194, 571-1024, 587-856, 594-841, 712-964, 712-974, 741-874, 775-1053, |
| | 776-1027 |
| 40/7505901CB1/ | 1-287, 3-281, 6-164, 21-276, 22-241, 24-471, 27-263, 27-321, 27-334, 27-1306, 28-286, 34-220, 37-281, 37-301, 37-342, |
| 1306 | 39-272, 40-316, 49-221, 54-179, 54-319, 55-299, 55-333, 55-336, 55-352, 56-300, 56-330, 57-266, 57-385, 57-404, |
| | 62-192, 62-221, 62-307, 75-310, 76-318, 76-358, 79-471, 82-296, 86-381, 102-387, 103-380, 130-374, 171-285, |
| | 177-453, 189-469, 194-430, 201-425, 202-322, 204-436, 204-471, 206-453, 206-469, 206-471, 208-322, 208-471, |
| | 233-341, 236-471, 237-471, 238-471, 238-500, 238-859, 278-471, 293-404, 293-416, 310-390, 325-542, 446-997, |
| | 468-725, 468-729, 468-737, 468-738, 468-785, 468-934, 468-951, 470-775, 471-736, 472-1056, 473-1277, 477-757, |
| | 482-743, 487-699, 487-734, 498-1059, 513-1057, 515-917, 515-966, 522-757, 529-794, 529-1128, 536-802, |
| | 538-1140, 544-1052, 550-754, 550-756, 551-770, 554-816, 559-1065, 565-696, 584-780, 585-811, 590-884, 592-839, |
| | 592-1164, 595-872, 607-873, 607-884, 609-1304, 615-785, 617-1306, 620-878, 623-890, 623-894, 629-919, |
| | 629-1304, 630-1306, 633-1242, 637-786, 649-908, 676-837, 676-1303, 677-1158, 677-1234, 689-870, 691-1018, |
| | 701-1006, 705-979, 714-1177, 715-977, 718-874, 725-1306, 729-990, 731-1276, 737-1306, 743-1009, 743-1010, |
| | 754-991, 759-970, 762-1052, 781-980, 781-1140, 781-1306, 802-1013, 823-1071, 825-1080, 855-1306, 857-1306, |
| | 858-1306, 871-1306, 873-1072, 873-1239, 873-1306, 874-1120, 874-1306, 875-1306, 879-1306, 880-1306, 884-1128, |
| | 884-1131, 886-1306, 898-1302, 904-1306, 910-1180, 912-1306, 915-1304, 917-1306, 919-1306, 921-1306, |
| | 922-1306, 927-1080, 927-1306, 930-1306, 937-1252, 937-1306, 941-1306, 946-1302, 952-1231, 955-1306, 956-1306, |
| | 962-1306, 980-1306, 982-1231, 985-1306, 991-1306, 996-1245, 998-1252, 999-1235, 1003-1259, 1003-1293, |
| | 1003-1306, 1013-1241, 1015-1287, 1018-1306, 1019-1306, 1020-1306, 1028-1095, 1033-1306, 1052-1240, 1064-1302, |
| | 1066-1306, 1068-1306, 1079-1306, 1087-1306, 1094-1305, 1096-1306, 1104-1225, 1104-1306, 1127-1306, |
| | 1153-1306, 1190-1306, 1204-1306, 1240-1306 |

TABLE 5

| Polynucleotide | Incyte | Representative |
|---|---|---|
| SEQ ID NO: | Project ID: | Library |
| 21 | 7500521CB1 | ADMEDNV37 |
| 22 | 7502992CB1 | COLNFET02 |
| 23 | 71187173CB1 | BRATDIC01 |
| 24 | 7503143CB1 | BRAITUT13 |
| 25 | 7503563CB1 | BMARTXE01 |
| 26 | 6244251CB1 | TESTNOC01 |
| 27 | 7503467CB1 | LUNGNON03 |
| 28 | 6599034CB1 | OVARNON03 |
| 29 | 7504179CB1 | PROSTUT10 |
| 30 | 71249354CB1 | LATRTUT02 |
| 31 | 7505803CB1 | TLYMUNT01 |
| 32 | 7505804CB1 | BRSTNOT19 |
| 33 | 7505846CB1 | EOSITXT01 |
| 34 | 55004585CB1 | BRAUTDR04 |
| 35 | 7506012CB1 | THYRNOT02 |
| 36 | 7506212CB1 | LUNGNOT10 |
| 37 | 7481808CB1 | TLYJTXF03 |
| 38 | 7488221CB1 | THYMNOE01 |
| 39 | 7505894CB1 | MLP000052 |
| 40 | 7505901CB1 | LEUKNOT03 |

TABLE 6

| Library | Vector | Library Description |
|---|---|---|
| ADMEDNV37 | PCR2- | Library was constructed using pooled cDNA from 111 different donors. cDNA was generated using mRNA isolated from |
| | TOPOTA | pooled skeletal muscle tissue removed from 10 Caucasian male and female donors, ages 21-57, who died from sudden |
| | | death; from pooled thymus tissue removed from 9 Caucasian male and female donors, ages 18-32, who died from sudden |
| | | death; from pooled fetal liver tissue removed from 32 Caucasian male and female fetuses, ages 18-24 weeks, who died |
| | | from spontaneous abortions; from pooled fetal kidney tissue removed from 59 Caucasian male and female fetuses, ages |
| | | 20-33 weeks, who died from spontaneous abortions; and from fetal brain tissue removed from a 23-week-old |
| | | Caucasian male fetus who died from fetal demise. |
| BMARTXE01 | pINCY | This 5′ biased random primed library was constructed using RNA isolated from treated SH-SY5Y cells derived from a |
| | | metastatic bone marrow neuroblastoma, removed from a 4-year-old Caucasian female (Schering AG). The medium was |
| | | MEM/HAM'S F12 with 10% fetal calf serum. After reaching about 80% confluency cells were treated with 6- |
| | | Hydroxydopamine (6-OHDA) at 100 microM for 8 hours. |
| BRAITUT13 | pINCY | Library was constructed using RNA isolated from brain tumor tissue removed from the left frontal lobe of a 68-year-old |
| | | Caucasian male during excision of a cerebral meningeal lesion. Pathology indicated a meningioma in the left frontal lobe. |
| BRATDIC01 | pINCY | This large size-fractionated library was constructed using RNA isolated from diseased brain tissue removed from the left |
| | | temporal lobe of a 27-year-old Caucasian male during a brain lobectomy. Pathology for the left temporal lobe, including |
| | | the mesial temporal structures, indicated focal, marked pyramidal cell loss and gliosis in hippocampal sector CA1, |
| | | consistent with mesial temporal sclerosis. The left frontal lobe showed a focal deep white matter lesion, characterized by |
| | | marked gliosis, calcifications, and hemosiderin-laden macrophages, consistent with a remote perinatal injury. The frontal |
| | | lobe tissue also showed mild to moderate generalized gliosis, predominantly subpial and subcortical, consistent with |
| | | chronic seizure disorder. GFAP was positive for astrocytes. The patient presented with intractable epilepsy, focal |
| | | epilepsy, hemiplegia, and an unspecified brain injury. Patient history included cerebral palsy, abnormality of |
| | | gait, depressive disorder, and tobacco abuse in remission. Previous surgeries included tendon transfer. |
| | | Patient medications included minocycline hydrochloride, Tegretol, phenobarbital, vitamin C, Pepcid, and Pevaryl. |
| | | Family history included brain cancer in the father. |
| BRAUTDR04 | PCDNA2.1 | This random primed library was constructed using RNA isolated from pooled striatum, dorsal caudate nucleus, dorsal |
| | | putamen, and ventral nucleus accumbens tissue removed from a 55-year-old Caucasian female who died from |
| | | cholangiocarcinoma. Pathology indicated mild meningeal fibrosis predominately over the convexities, scattered axonal |
| | | spheroids in the white matter of the cingulate cortex and the thalamus, and a few scattered neurofibrillary tangles in the |
| | | entorhinal cortex and the periaqueductal gray region. Pathology for the associated tumor tissue indicated |
| | | well-differentiated cholangiocarcinoma of the liver with residual or relapsed tumor. Patient history |
| | | included cholangiocarcinoma, post-operative Budd-Chiari syndrome, biliary ascites, hydrothorax, dehydration, |
| | | malnutrition, oliguria and acute renal failure. Previous surgeries included cholecystectomy and |
| | | resection of 85% of the liver. |
| BRSTNOT19 | pINCY | Library was constructed using RNA isolated from breast tissue removed from a 67-year-old Caucasian female during a |
| | | unilateral extended simple mastectomy. Pathology for the associated tumor tissue indicated residual invasive lobular |
| | | carcinoma. Patient history included depressive disorder, benign large bowel neoplasm, and hemorrhoids. Family history |
| | | included cerebrovascular and cardiovascular disease and lung cancer. |
| COLNFET02 | pINCY | Library was constructed using RNA isolated from the colon tissue of a Caucasian female fetus, who died at 20 weeks' |
| | | gestation. |
| EOSITXT01 | pINCY | Library was constructed using RNA isolated from eosinophils stimulated with IL-5. |
| LATRTUT02 | pINCY | Library was constructed using RNA isolated from a myxoma removed from the left atrium of a 43-year-old |
| | | Caucasian male during annuloplasty. Pathology indicated atrial myxoma. Patient history included pulmonary |
| | | insufficiency, acute myocardial infarction, atherosclerotic coronary artery disease, hyperlipidemia, and tobacco |
| | | use. Family history included benign hypertension, acute myocardial infarction, atherosclerotic coronary artery |
| | | disease, and type II diabetes. |
| LEUKNOT03 | pINCY | Library was constructed using RNA isolated from white blood cells of a 27-year-old female with blood type A+. |
| | | The donor tested negative for cytomegalovirus (CMV). |
| LUNGNON03 | PSPORT1 | This normalized library was constructed from 2.56 million independent clones from a lung tissue library. RNA was made |
| | | from lung tissue removed from the left lobe of a 58-year-old Caucasian male during a segmental lung resection. |
| | | Pathology for the associated tumor tissue indicated a metastatic grade 3 (of 4) osteosarcoma. Patient history |
| | | included soft tissue cancer, secondary cancer of the lung, prostate cancer, and an acute duodenal ulcer with |
| | | hemorrhage. Patient also received radiation therapy to the retroperitoneum. Family history included prostate |
| | | cancer, breast cancer, and acute leukemia. The normalization and hybridization conditions were adapted from |
| | | Soares et al., PNAS (1994) 91: 9228; Swaroop et al., NAR (1991) 19: 1954; and Bonaldo et al., |
| | | Genome Research (1996) 6: 791. |
| LUNGNOT10 | pINCY | Library was constructed using RNA isolated from the lung tissue of a Caucasian male fetus, who died at 23 weeks' |
| | | gestation. |
| MLP000052 | PCR2- | Library was constructed using pooled cDNA from different donors. cDNA was generated using mRNA isolated from the |
| | TOPOTA | following: aorta, cerebellum, lymph nodes, muscle, tonsil (lymphoid hyperplasia), bladder tumor (invasive grade 3 |
| | | transitional cell carcinoma.), breast (proliferative fibrocystic changes without atypia characterized by epithelial ductal |
| | | hyperplasia, testicle tumor (embryonal carcinoma), spleen, ovary, parathyroid, ileum, breast skin, sigmoid colon, penis |
| | | tumor (fungating invasive grade 4 squamous cell carcinoma), fetal lung,, breast, fetal small intestine, fetal liver, fetal |
| | | pancreas, fetal lung, fetal skin, fetal penis, fetal bone, fetal ribs, frontal brain tumor (grade 4 gemistocytic astrocytoma), |
| | | ovary (stromal hyperthecosis), bladder, bladder tumor (invasive grade 3 transitional cell carcinoma), stomach, |
| | | lymph node tumor (metastatic basaloid squamous cell carcinoma), tonsil (reactive lymphoid hyperplasia), periosteum |
| | | from the tibia, fetal brain, fetal spleen, uterus tumor, endometrial (grade 3 adenosquamous carcinoma), |
| | | seminal vesicle, liver, aorta, adrenal gland, lymph node (metastatic grade 3 squamous cell carcinoma), |
| | | glossal muscle, esophagus, esophagus tumor (invasive grade 3 adenocarcinoma), ileum, pancreas, soft tissue |
| | | tumor from the skull (grade 3 ependymoma), transverse colon, (benign familial polyposis), rectum tumor (grade 3 |
| | | colonic adenocarcinoma), rib tumor, (metastatic grade 3 osteosarcoma), lung, heart, placenta, thymus, stomach, |
| | | spleen (splenomegaly with congestion), uterus, cervix (mild chronic cervicitis with focal squamous |
| | | metaplasia), spleen tumor (malignant lymphoma, diffuse large cell type, B-cell phenotype with |
| | | abundant reactive T-cells and marked granulomatous response), umbilical cord blood mononuclear cells, |
| | | upper lobe lung tumor, (grade 3 squamous cell carcinoma), endometrium (secretory phase), liver, liver tumor |
| | | (metastatic grade 2 neuroendocrine carcinoma), colon, umbilical cord blood, Th1 cells, nonactivated, umbilical cord |
| | | blood, Th2 cells, nonactivated, coronary artery endothelial cells (untreated), coronary artery smooth muscle cells, |
| | | (untreated), coronary artery smooth muscle cells (treated with TNF & IL-1 10 ng/ml each for 20 hours), |
| | | bladder (mild chronic cystitis), epiglottis, breast skin, small intestine, fetal prostate stroma fibroblasts, |
| | | prostate epithelial cells (PrEC cells), fetal adrenal glands, fetal liver, kidney transformed embryonal cell |
| | | line (293-EBNA) (untreated), kidney transformed embryonal cell line (293-EBNA) (treated with 5Aza-2deoxycytidine |
| | | for 72 hours), mammary epithelial cells, (HMEC cells), peripheral blood monocytes (treated with |
| | | IL-10 at time 0, 10 ng/ml, LPS was added at 1 hour at 5 ng/ml. Incubation 24 hours), peripheral blood |
| | | monocytes (treated with anti-IL-10 at time 0, 10 ng/ml, LPS was added at 1 hour at 5 ng/ml. |
| | | Incubation 24 hours), spinal cord, base of medulla (Huntington's chorea), thigh and arm muscle (ALS), breast skin |
| | | fibroblast (untreated), breast skin fibroblast (treated with 9CIS Retinoic Acid 1 μM for 20 hours), breast skin fibroblast |
| | | (treated with TNF-alpha & IL-1 beta, 10 ng/ml each for 20 hours), fetal liver mast cells, hematopoietic (Mast cells |
| | | prepared from human fetal liver hematopoietic progenitor cells (CD34+ stem cells) cultured in the presence of |
| | | hIL-6 and hSCF for 18 days), epithelial layer of colon, bronchial epithelial cells (treated for 20 hours with |
| | | 20% smoke conditioned media), lymph node, pooled peripheral blood mononuclear cells (untreated), pooled brain |
| | | segments: striatum, globus pallidus and posterior putamen (Alzheimer's Disease), pituitary gland, umbilical |
| | | cord blood, CD34+ derived dendritic cells (treated with SCF, GM-CSF & TNF alpha, 13 days), umbilical cord |
| | | blood, CD34+ derived dendritic cells (treated with SCF, GM-CSF & TNF alpha, 13 days followed by |
| | | PMA/Ionomycin for 5 hours), small intestine, rectum, bone marrow neuroblastoma cell line (SH-SY5Y cells, |
| | | treated with 6-Hydroxydopamine 100 uM for 8 hours), bone marrow, neuroblastoma cell |
| | | line (SH-SY5Y cells, untreated), brain segments from one donor: amygdala, entorhinal cortex, globus |
| | | pallidus, substantia innominata, striatum, dorsal caudate nucleus, dorsal putamen, ventral nucleus accumbens, |
| | | archaecortex (hippocampus anterior and posterior), thalamus, nucleus raphe magnus, periaqueductal gray, |
| | | midbrain, substantia nigra, and dentate nucleus, pineal gland (Alzheimer's Disease), preadipocytes (untreated), |
| | | preadipocytes (treated with a peroxisome proliferator-activated receptor gamma agonist, 1microM, 4 hours), |
| | | pooled prostate (adenofibromatous hyperplasia), pooled kidney, pooled adipocytes (untreated), pooled |
| | | adipocytes (treated with human insulin), pooled mesentaric and abdomenal fat, pooled adrenal glands, pooled |
| | | thyroid (normal and adenomatous hyperplasia), pooled spleen (normal and with changes consistent |
| | | with idiopathic thrombocytopenic purpura), pooled right and left breast pooled lung, pooled nasal |
| | | polyps, pooled fat, pooled synovium (normal and rhumatoid arthritis), pooled brain (meningioma, |
| | | gemistocytic astrocytoma. and Alzheimer's disease), pooled fetal colon, pooled colon: ascending, descending (chronic |
| | | ulcerative colitis), and rectal tumor (adenocarcinoma), pooled esophagus, normal and tumor (invasive grade 3 |
| | | adenocarcinoma), pooled breast skin fibroblast (one treated w/9CIS Retinoic Acid and the other with TNF-alpha & IL-1 |
| | | beta), pooled gallbladder (acute necrotizing cholecystitis with cholelithiasis (clinically hydrops), acute hemorrhagic |
| | | cholecystitis with cholelithiasis, chronic cholecystitis and cholelithiasis), pooled fetal heart, (Patau's and fetal demise), |
| | | pooled neurogenic tumor cell line, SK-N-MC, (neuroepitelioma, metastasis to supra-orbital area, untreated) and neuron, |
| | | NT-2 cell line, (treated with mouse leptin at 1 μg/ml and 9cis retinoic acid at 3.3 μM for 6 days), pooled ovary (normal |
| | | and polycystic ovarian disease), pooled prostate, (adenofibromatous hyperplasia), pooled seminal vesicle, pooled small |
| | | intestine, pooled fetal small intestine, pooled stomach and fetal stomach, prostate epithelial cells, pooled testis (normal |
| | | and embryonal carcinoma), pooled uterus, pooled uterus tumor (grade 3 adenosquamous carcinoma and leiomyoma), |
| | | pooled uterus, endometrium, and myometrium, (normal and adenomatous hyperplasia with squamous metaplasia and |
| | | focal atypia), pooled brain: (temporal lobe meningioma, cerebellum and hippocampus |
| | | (Alzheimer's Disease), pooled skin, fetal lung, |
| | | adrenal tumor (adrenal cortical carcinoma), prostate |
| | | tumor (adenocarcinoma), fetal heart, fetal small intestine, ovary tumor |
| | | (mucinous cystadenoma), ovary, ovary tumor (transitional cell carcinoma), disease prostate (adenofibromatous |
| | | hyperplasia), fetal colon, uterus tumor (leiomyoma), temporal brain, |
| | | submandibular gland, colon tumor (adenocarcinoma), ascending and transverse |
| | | colon, ovary tumor (endometrioid carcinoma), lung tumor (squamous cell carcinoma), fetal brain, |
| | | fetal lung, ureter tumor (transitional cell carcinoma), untreated HNT cells, para-aortic soft tissue, testis, |
| | | seminal vesicle, diseased ovary (endometriosis), temporal lobe, myometrium, diseased gallbladder (cholecystitis, |
| | | cholelithiasis), placenta, breast tumor (ductal adenocarcinoma), breast, |
| | | lung tumor (liposarcoma), endometrium, abdominal fat, cervical spine |
| | | dorsal root ganglion, thoracic spine dorsal root ganglion, diseased thyroid (adenomatous hyperplasia), |
| | | liver, kidney, fetal liver, NT-2 cells (treated with mouse leptin and 9cis RA), K562 cells (treated with 9cis RA), |
| | | cerebellum, corpus callosum, hypothalamus, fetal brain astrocytes (treated with TNFa and IL-1b), inferior parietal cortex, |
| | | posterior hippocampus, pons, thalamus, C3A cells (untreated), |
| | | C3A cells (treated with 3-methylcholanthrene), testis, colon |
| | | epithelial layer, pooled prostate, pooled liver, substantia nigra, thigh muscle, rib bone, fallopian tube tumor (endometrioid |
| | | and serous adenocarcinoma), diseased lung (idiopathic pulmonary disease), cingulate anterior allocortex and neocortex, |
| | | cingulate posterior allocortex, auditory neocortex, frontal neocortex, |
| | | orbital inferior neocortex, parietal superior neocortex, |
| | | visual primary neocortex, dentate nucleus, posterior cingulate, cerebellum, vermis, inferior temporal |
| | | cortex, medulla, posterior parietal cortex, colon polyp, pooled breast, anterior and posterior hippocampus, mesenteric and |
| | | abdominal fat, pooled esophagus, pooled fetal kidney, |
| | | pooled fetal liver, ileum, small intestine, pooled gallbladder, frontal |
| | | and superior temporal cortex, pooled ovary, pooled endometrium, pooled prostate, pooled kidney, fetal femur, sacrum |
| | | tumor (giant cell tumor), pooled kidney and kidney tumor (renal cell carcinoma clear-cell type), pooled liver and liver |
| | | tumor (neuroendocrine carcinoma), pooled fetal liver, pooled lung, fetal pancreas, pancreas, parotid gland, parotid tumor |
| | | (sebaceous lymphadenoma), retroperitoneal and suprglottic soft tissue, spleen, fetal spleen, spleen tumor (malignant |
| | | lymphoma), diseased spleen (idiopathic thrombocytopenic purpura), parathyroid, thyroid, thymus, tonsil ureter tumor |
| | | (transitional cell carcinoma), pooled adrenal gland and adrenal tumor (pheochromocytoma), pooled lymph node tumor |
| | | (Hodgkin's disease and metastatic adenocarcinoma), pooled neck and calf muscles, and pooled bladder. |
| OVARNON03 | pINCY | This normalized ovarian tissue library was constructed from 5 million independent clones from an |
| | | ovary library. Starting RNA was made from ovarian tissue removed from a 36-year-old Caucasian female during total |
| | | abdominal hysterectomy, bilateral salpingo-oophorectomy, soft tissue excision, and an incidental appendectomy. |
| | | Pathology for the associated tumor tissue indicated one intramural and one subserosal leiomyomata of the myometrium. |
| | | The endometrium was proliferative phase. Patient history included |
| | | deficiency anemia, calculus of the kidney, and a kidney |
| | | anomaly. Family history included hyperlipidemia, acute myocardial infarction, atherosclerotic coronary artery disease, |
| | | type II diabetes, and chronic liver disease. The library was normalized in two rounds using conditions adapted from |
| | | Soares et al., PNAS (1994) 91: 9228 and Bonaldo et al., Genome Research (1996) 6: 791, except that a significantly |
| | | longer (48 hours/round) reannealing hybridization was used. |
| PROSTUT10 | pINCY | Library was constructed using RNA isolated from prostatic tumor tissue removed from a 66-year-old Caucasian male |
| | | during radical prostatectomy and regional lymph node excision. Pathology indicated an adenocarcinoma (Gleason grade |
| | | 2 + 3). Adenofibromatous hyperplasia was also present. The patient presented with elevated |
| | | prostate specific antigen (PSA). Family history included prostate cancer and secondary bone cancer. |
| TESTNOC01 | PBLUE- | This large size fractionated library was constructed using RNA isolated from testicular tissue removed from a pool of |
| | SCRIPT | eleven, 10 to 61-year-old Caucasian males. |
| THYMNOE01 | PCDNA2.1 | This 5′ biased random primed library was constructed using RNA isolated from thymus tissue removed from a 2-year-old |
| | | Caucasian female during a thymectomy and patch closure of left atrioventricular fistula. Pathology indicated there was no |
| | | gross abnormality of the thymus. The patient presented with congenital heart abnormalities. Patient history |
| | | included double inlet left ventricle and a rudimentary right ventricle, pulmonary hypertension, cyanosis, |
| | | subaortic stenosis, seizures, and a fracture of the skull base. Patient medications included Lasix and |
| | | Captopril. Family history included reflux neuropathy in the mother. |
| THYRNOT02 | PSPORT1 | Library was constructed using RNA isolated from the diseased thyroid tissue of a 16-year-old Caucasian female with |
| | | Graves' disease (hyperthyroidism). |
| TLYJTXF03 | pRARE | This 5′ cap isolated full-length library was constructed using RNA isolated from a treated Jurkat cell line derived from the |
| | | T cells of a male. The cells were treated with 10 ng/mL of anti-CD3 for 5 minutes. The cells were then fractionated to |
| | | obtain the nuclei. Patient history included acute T-cell leukemia. |
| TLYMUNT01 | pINCY | Library was constructed using RNA isolated from resting allogenic T-lymphocyte tissue removed from an |
| | | adult (40-50-year old) Caucasian male. |

TABLE 7

| | | | Parameter |
|---|---|---|---|
| Program | Description | Reference | Threshold |
| ABI | A program that removes vector sequences and | Applied Biosystems, Foster City, CA. | |
| FACTURA | masks ambiguous bases in nucleic acid sequences. | | |
| ABI/ | A Fast Data Finder useful in comparing and | Applied Biosystems, Foster City, CA; | Mismatch |
| PARACEL | annotating amino acid or nucleic acid sequences. | Paracel Inc., Pasadena, CA. | <50% |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: |
| | sequence similarity search for amino acid and | 215: 403-410; Altschul, S. F. et al. (1997) | Probability |
| | nucleic acid sequences. BLAST includes five | Nucleic Acids Res. 25: 3389-3402. | value = 1.0E−8 |
| | functions: blastp, blastn, blastx, tblastn, and tblastx. | | or less; Full |
| | | | Length |
| | | | sequences: |
| | | | Probability |
| | | | value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, | value = |
| | sequences of the same type. FASTA comprises as | W. R. (1990) Methods Enzymol. 183: 63-98; | 1.06E−6; |
| | least five functions: fasta, tfasta, fastx, tfastx, and | and Smith, T. F. and M. S. Waterman (1981) | Assembled |
| | ssearch. | Adv. Appl. Math. 2: 482-489. | ESTs: fasta |
| | | | Identity = 95% |
| | | | or greater and |
| | | | Match length = |
| | | | 200 bases or |
| | | | greater; fastx E |
| | | | value = 1.0E−8 |
| | | | or less; Full |
| | | | Length |
| | | | sequences: |
| | | | fastx score = |
| | | | 100 or greater |
| BLIMPS | A BLocks IMProved Searcher that matches a | Henikoff, S. and J. G. Henikoff (1991) Nucleic | Probability |
| | sequence against those in BLOCKS, PRINTS, | Acids Res. 19: 6565-6572; Henikoff, J. G. and | value = 1.0E−3 |
| | DOMO, PRODOM, and PFAM databases to search | S. Henikoff (1996) Methods Enzymol. | or less |
| | for gene families, sequence homology, and structural | 266: 88-105; and Attwood, T. K. et al. (1997) J. | |
| | fingerprint regions. | Chem. Inf. Comput. Sci. 37: 417-424. | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART or |
| | protein family consensus sequences, such as PFAM, | (1988) Nucleic Acids Res. 26: 320-322; | TIGRFAM |
| | INCY, SMART and TIGRFAM. | Durbin, R. et al. (1998) Our World View, in a | hits: |
| | | Nutshell, Cambridge Univ. Press, pp. 1-350. | Probability |
| | | | value = 1.0E−3 |
| | | | or less; |
| | | | Signal peptide |
| | | | hits: Score = 0 |
| | | | or greater |
| ProfileScan | An algorithm that searches for structural and sequence | Gribskov, M. et al. (1988) CABIOS 4: 61-66; | Normalized |
| | motifs in protein sequences that match sequence patterns | Gribskov, M. et al. (1989) Methods Enzymol. | quality score ≧ |
| | defined in Prosite. | 183: 146-159; Bairoch, A. et al. (1997) | GCG-specified |
| | | Nucleic Acids Res. 25: 217-221. | “HIGH” value |
| | | | for that |
| | | | particular |
| | | | Prosite motif. |
| | | | Generally, |
| | | | score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or |
| | CrossMatch, programs based on efficient implementation | Appl. Math. 2: 482-489; Smith, T. F. and M. S. | greater; |
| | of the Smith-Waterman algorithm, useful in searching | Waterman (1981) J. Mol. Biol. 147: 195-197; | Match length = |
| | sequence homology and assembling DNA sequences. | and Green, P., University of Washington, | 56 or greater |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = 3.5 or |
| | sequences for the presence of secretory signal peptides. | 10: 1-6; Claverie, J. M. and S. Audic (1997) | greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. On Intelligent Systems for Mol. Biol., | |
| | and determine orientation. | Glasgow et al., eds., The Am. Assoc. for Artificial | |
| | | Intelligence (AAAI) Press, Menlo Park, CA, and MIT | |
| | | Press, Cambridge, MA pp. 175-182. | |
| Motifs | A program that searches amino acid sequences for patterns | Bairoch, A. et al. (1997) Nucleic Acids | |
| | that matched those defined in Prosite. | Res. 25: 217-221; | |
| | | Wisconsin Package Program Manual, version 9, page | |
| | | M51-59, Genetics Computer Group, Madison, WI. | |

TABLE 8

| | | | | | | | | | | Cau- | | | |
|---|---|---|---|---|---|---|---|---|---|---|---|---|---|
| | | | | | | | | | | casian | African | Asian | Hispanic |
| SEQ | | | | | | | | | | Allele 1 | Allele 1 | Allele 1 | Allele 1 |
| ID | | | | EST | CB1 | EST | Allele | Allele | Amino | fre- | fre- | fre- | fre- |
| NO: | PID | EST ID | SNP ID | SNP | SNP | Allele | 1 | 2 | Acid | quency | quency | quency | quency |

| | | | | | | | | | | | | | |
|---|---|---|---|---|---|---|---|---|---|---|---|---|---|
| 36 | 7506212 | 1334172H1 | SNP00001710 | 124 | 174 | C | G | C | A40 | n/a | n/a | n/a | n/a |
| 36 | 7506212 | 1342069H1 | SNP00015140 | 344 | 563 | C | C | G | non- | n/a | n/a | n/a | n/a |
| | | | | | | | | | coding | | | | |
| 36 | 7506212 | 1342069H1 | SNP00015141 | 82 | 4611 | A | A | C | non- | 0.92 | n/a | n/a | n/a |
| | | | | | | | | | coding | | | | |
| 36 | 7506212 | 1376878H1 | SNP00019042 | 201 | 4198 | T | T | C | non- | n/d | n/a | n/a | n/a |
| | | | | | | | | | coding | | | | |
| 36 | 7506212 | 1413622H1 | SNP00146236 | 65 | 4948 | G | G | C | non- | n/a | n/a | n/a | n/a |
| | | | | | | | | | coding | | | | |
| 36 | 7506212 | 1990335H1 | SNP00003545 | 89 | 3544 | A | G | A | V1163 | 0.33 | 0.30 | 0.37 | 0.49 |
| 36 | 7506212 | 2097343H1 | SNP00023386 | 82 | 3287 | G | G | A | E1078 | n/a | n/a | n/a | n/a |
| 36 | 7506212 | 2444202H1 | SNP00112338 | 213 | 2084 | G | G | C | G677 | n/d | n/d | n/d | n/d |
| 36 | 7506212 | 3373271H1 | SNP00019041 | 88 | 4015 | T | T | C | non- | n/a | n/a | n/a | n/a |
| | | | | | | | | | coding | | | | |
| 36 | 7506212 | 5029814H1 | SNP00063493 | 38 | 1719 | A | A | G | T222 | 0.89 | n/a | n/a | n/a |
| 38 | 7488221 | 1287507H1 | SNP00143006 | 216 | 580 | A | A | C | K187 | n/a | n/a | n/a | n/a |
| 38 | 7488221 | 1643522H1 | SNP00000508 | 71 | 2704 | C | C | A | non- | 0.87 | 0.79 | 0.93 | 0.76 |
| | | | | | | | | | coding | | | | |
| 38 | 7488221 | 3487206H1 | SNP00000508 | 42 | 2702 | A | C | A | non- | 0.87 | 0.79 | 0.93 | 0.76 |
| | | | | | | | | | coding | | | | |
| 38 | 7488221 | 3973154H1 | SNP00000508 | 35 | 2703 | C | C | A | non- | 0.87 | 0.79 | 0.93 | 0.76 |
| | | | | | | | | | coding | | | | |
| 38 | 7488221 | 5290303H1 | SNP00000509 | 123 | 3373 | T | T | C | non- | n/a | n/a | n/a | n/a |
| | | | | | | | | | coding | | | | |
| 38 | 7488221 | 5464541H1 | SNP00143006 | 95 | 579 | A | A | C | K187 | n/a | n/a | n/a | n/a |
| 38 | 7488221 | 5796955H1 | SNP00000509 | 440 | 3375 | T | T | C | non- | n/a | n/a | n/a | n/a |
| | | | | | | | | | coding | | | | |
| 38 | 7488221 | 6205687H1 | SNP00132515 | 63 | 1163 | C | T | C | H382 | n/a | n/a | n/a | n/a |
| 39 | 7505894 | 2688406H1 | SNP00061373 | 113 | 841 | T | T | C | non- | n/a | n/a | n/a | n/a |
| | | | | | | | | | coding | | | | |
| 39 | 7505894 | 5802504H1 | SNP00061373 | 60 | 832 | T | T | C | non- | n/a | n/a | n/a | n/a |
| | | | | | | | | | coding | | | | |
| 40 | 7505901 | 1723825H1 | SNP00041209 | 111 | 983 | G | G | C | K236 | n/a | n/a | n/a | n/a |
| 40 | 7505901 | 405880H1 | SNP00041209 | 100 | 984 | G | G | C | E237 | n/a | n/a | n/a | n/a |
| 40 | 7505901 | 4435683H1 | SNP00041209 | 239 | 982 | G | G | C | R236 | n/a | n/a | n/a | n/a |
| 40 | 7505901 | 6870884H1 | SNP00041209 | 236 | 986 | G | G | C | K237 | n/a | n/a | n/a | n/a |

1. An isolated polypeptide selected from the group consisting of:
a) a polypeptide comprising an amino acid sequence selected from the group consisting of SEQ ID NO:1-20,
b) a polypeptide comprising a naturally occurring amino acid sequence at least 90% identical to an amino acid sequence selected from the group consisting of SEQ ID NO:1, SEQ ID NO:2, SEQ ID NO:7-9, SEQ ID NO:14, and SEQ ED NO: 17,
c) a polypeptide comprising a naturally occurring amino acid sequence at least 92% identical to the amino acid sequence of SEQ ID NO:6,
d) a polypeptide comprising a naturally occurring amino acid sequence at least 94% identical to the amino acid sequence of SEQ ID NO:18,
e) a polypeptide comprising a naturally occurring amino acid sequence at least 95% identical to the amino acid sequence of SEQ ID NO:5,
f) a polypeptide comprising a naturally occurring amino acid sequence at least 96% identical to the amino acid sequence of SEQ ID NO:10,
g) a polypeptide comprising a naturally occurring amino acid sequence at least 98% identical to the amino acid sequence of SEQ ID NO:4,
h) a polypeptide consisting essentially of a naturally occurring amino acid sequence at least 90% identical to an amino acid sequence selected from the group consisting of SEQ ID NO:11-13, SEQ ID NO:15, SEQ ID NO:16, SEQ ID NO:19, and SEQ ID NO:20,
i) a biologically active fragment of a polypeptide having an amino acid sequence selected from the group consisting of SEQ ID NO:1-20, and
j) an immunogenic fragment of a polypeptide having an amino acid sequence selected from the group consisting of SEQ ID NO:1-20.

2. An isolated polypeptide of claim 1 comprising an amino acid sequence selected from the group consisting of SEQ ID NO:1-20.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:21-40.

6. A recombinant polynucleotide comprising a promoter sequence operably linked to a polynucleotide of claim 3.

7. A cell transformed with a recombinant polynucleotide of claim 6.

8. (CANCELLED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-20.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:21-40,
b) a polynucleotide comprising a naturally occurring polynucleotide sequence at least 90% identical to a polynucleotide sequence selected from the group consisting of SEQ ID NO:21, SEQ ID NO:22, and SEQ ID NO:25-39,
c) a polynucleotide comprising a naturally occurring polynucleotide sequence at least 97% identical to the polynucleotide sequence of SEQ ID NO:24,
d) a polynucleotide comprising a naturally occurring polynucleotide sequence at least 98% identical to the polynucleotide sequence of SEQ ID NO:40,
e) a polynucleotide complementary to a polynucleotide of a),
f) a polynucleotide complementary to a polynucleotide of b),
g) a polynucleotide complementary to a polynucleotide of c),
h) a polynucleotide complementary to a polynucleotide of d), and
i) an RNA equivalent of a)-h).

13. (CANCELLED)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELLED)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-20.

19. (CANCELLED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (CANCELLED)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-25. (CANCELLED)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELLED)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30.-95. (CANCELLED)
